(19) United States
(12) Patent Application Publication
(10) Pub. No.: US 2020/0138878 A1
(43) Pub. Date: May 7, 2020

(54) PROBIOTIC-BASED TREATMENT OF RESISTANT CHRONIC RHINOSINUSITIS

(71) Applicant: PROBIONASE THERAPIES INC., Outremont (CA)

(72) Inventor: Martin DESROSIERS, Outremont (CA)

(21) Appl. No.: 16/628,888

(22) PCT Filed: Jul. 6, 2017
(86) PCT No.: PCT/CA2017/050814

§ 371 Date: Jan. 6, 2020

(51) Int. Cl. A61K 35/744; A61K 31/58; A61K 9/00; A61P 11/02

(52) U.S. Cl. CPC A61K 35/744; A61P 11/02; A61K 9/0043; A61K 31/58

(57) ABSTRACT

Methods, uses, compositions and kits for treating steroid-resistant chronic rhinosinusitis (CRS), including refractory CRS, in a subject, or for improving or restoring the response to anti-inflammatory sinus treatment in a patient suffering from steroid-resistant CRS, are described. These methods, uses, compositions and kits are based on the sinus administration of an effective amount of probiotics, such as lactic acid bacteria (LAB), for example Lactococcus lactis bacteria.

### TECHNICAL FIELD

The present invention generally relates to the management of chronic rhinosinusitis (CRS), and more particularly to the treatment of CRS resistant to conventional medical and/or surgical treatment, such as steroid treatment.

### BACKGROUND ART

The symptoms of rhinosinusitis include nasal congestion and obstruction, colored nasal discharge, anterior or posterior nasal drip. Subjects may also experience facial pain or pressure, and suffer a reduction or a loss of smell (hyposmia; anosmia). There are two different types of rhinosinusitis: acute and chronic. Acute rhinosinusitis is characterized as rhinosinusitis with complete resolution of symptoms within 12 weeks, while chronic rhinosinusitis (CRS) lasts longer than 12 weeks, and usually involves tissue damage. Nasal polyps are frequently present in some subjects with chronic rhinosinusitis based on epidemiologic studies.

CRS is the second most common chronic disease (16%) and affected patients suffer serious discomfort, inconvenience and chronic and painful infection, which impact negatively on quality of life, productivity and health costs^{1,2}. Co-morbid disorders such as asthma and sleep-disordered breathing may also be negatively impacted³⁻⁵. CRS is considered a complex disease, where multiple factors, including inflammatory cell infiltrate, microbiome dysbiosis, and dysfunction of the barrier function of the sinus epithelium interact to initiate and maintain the clinical phenotypes of chronic rhinosinusitis with nasal polyposis (CRSwNP) and chronic rhinosinusitis without nasal polyps (CRSsNP)^{1,6-8}.

There is a need to develop new therapies for chronic rhinosinusitis. The aims of treatment in CRS include elimination of the infection, reduced sinonasal inflammation, and restoration of sinonasal passage drainage patency. Conventional medical treatment includes saline washes, anti-inflammatory agents (e.g., corticosteroids), and antibiotics, but there are significant proportion of patients for whom such treatment, notably steroids, are ineffective. Surgery is usually reserved for patients not responding to medical therapy. Currently, even following surgery, endoscopic signs of recurrence are seen in over 50% of the patients by four months after surgery, and even postoperative therapy with topical corticosteroid drops or sprays does not prevent the recurrence of disease.^{1,9} These patients are deemed to suffer from CRS refractory to medical and surgical therapy, and represent a considerable burden to both affected patients and the health care system. Also, long-term use of corticosteroids and antibiotics may lead to adverse effects, drug interactions, and antimicrobial resistance. Novel alternate therapies are thus required.

Mukerji et al. (Otolaryngology—Head and Neck Surgery, vol. 140, no. 2, pp. 202-208, 2009) analysed the oral use of the probiotic Lactobacillus rhamnosus on sinonasal quality-of-life scores in 77 CRS patients, and concluded that a 4-week oral probiotic treatment led to no significant results relative to placebo.

The present description refers to a number of documents, the content of which is herein incorporated by reference in their entirety.

### SUMMARY OF THE INVENTION

The present invention provides the following items 1 to 64:

1. A method of treating a patient suffering from steroid-resistant chronic rhinosinusitis (CRS), said method comprising administering to a sinus from said subject (i) a composition comprising an effective amount of probiotics; and (ii) an effective amount of an anti-inflammatory agent.
2. A method of improving or restoring the response to anti-inflammatory sinus treatment in a patient suffering from steroid-resistant chronic rhinosinusitis (CRS), said method comprising administering to a sinus from said subject a composition comprising an effective amount of probiotics.
3. The method of item 1 or 2, wherein said anti-inflammatory agent is a steroid.
4. The method of item 3, wherein said steroid is a corticosteroid.
5. The method of item 4, wherein said corticosteroid is budenoside.
6. The method of any one of items 1 to 5, wherein the administering is by sinus rinse or irrigation.
7. The method of any one of items 1 to 6, wherein said composition comprises a mixture of different probiotic strains.
8. The method of any one of items 1 to 6, wherein said composition comprises a single probiotic strain.
9. The method of any one of items 1 to 8, wherein said probiotics comprise Gram-positive bacteria.
10. The method of item 9, wherein said Gram-positive bacteria are lactic acid bacteria (LAB).
11. The method of item 10, wherein said LAB is of the Lactococcus genus.
12. The method of item 11, wherein said LAB of the Lactococcus genus is Lactococcus lactis,
13. The method of item 12, wherein said Lactococcus lactis is Lactococcus lactis subsp. cremoris.
14. The method of any one of items 1 to 13, wherein the composition comprising probiotics is administered twice-daily.
15. The method of any one of items 1 to 14, wherein the composition comprising probiotics is administered for a period of at least two weeks.
16. The method of item 15, wherein the composition comprising probiotics is administered for a period of about 3 to 5 weeks.
17. The method of any one of items 1 to 16, wherein the probiotics are present in an amount corresponding to at least about 10⁶ colony-forming units (CFU) in said composition.
18. The method of item 17, wherein the probiotics are present in an amount corresponding to about 10⁸ to about 10¹² CFU in said composition.
19. The method of any one of items 1 to 18, wherein the composition comprising probiotics is a liquid composition and comprises a concentration of probiotics of at least about 10⁵ CFU/mL.
20. The method of item 19, wherein the concentration of probiotics is about 10⁶ to about 10⁸ CFU/mL.
21. The method of item 19 or 20, wherein the liquid composition comprises a saline buffer.
22. The method of item 21, wherein said method further comprises resuspending said probiotics in said saline buffer prior to administration.
23. The method of any one of items 1 to 22, wherein said probiotics and said anti-inflammatory agent are in different compositions.
24. The method of any one of items 1 to 23, wherein said method improves at least one of the following CRS symptom parameters in said subject: sino-nasal symptom score (SNSS), Sino-nasal Outcome test 22 items (SNOT-22) score and/or peri-operative sinus endoscopy (POSE) score.
25. The method of item 24, wherein said method improves all of the following CRS symptom parameters in said subject: SNSS, SNOT-22 score, and POSE score.
26. The method of any one of items 1 to 25, wherein said steroid-resistant CRS is steroid-resistant CRS with nasal polyps.
27. The method of any one of items 1 to 26, wherein said steroid-resistant CRS is refractory CRS.
28. Use of (i) a composition comprising an effective amount of probiotics; and (ii) an effective amount of an anti-inflammatory agent, for treating a patient suffering from steroid-resistant CRS, wherein said composition comprising an effective amount of probiotics and said anti-inflammatory agent are for sinus administration.
29. Use of (i) a composition comprising an effective amount of probiotics; and (ii) an effective amount of an anti-inflammatory agent, for the manufacture of a medicament for treating a patient suffering from steroid-resistant CRS, wherein said composition comprising an effective amount of probiotics and said anti-inflammatory agent are for sinus administration.
30. A combination for treating a patient suffering from steroid-resistant CRS, said combination comprising (i) a composition comprising an effective amount of probiotics; and (ii) an effective amount of an anti-inflammatory agent, wherein said composition comprising an effective amount of probiotics and said anti-inflammatory agent are for sinus administration.
31. Use of a composition comprising an effective amount of probiotics for improving or restoring the response to anti-inflammatory sinus treatment in a patient suffering from steroid-resistant CRS, wherein said composition is for sinus administration.
32. Use of a composition comprising an effective amount of probiotics for the manufacture of a medicament for improving or restoring the response to anti-inflammatory sinus treatment in a patient suffering from steroid-resistant CRS, wherein said composition is for sinus administration.
33. A composition comprising an effective amount of probiotics for improving or restoring the response to anti-inflammatory sinus treatment in a patient suffering from steroid-resistant CRS, wherein said composition is for sinus administration.
34. The use or composition for use according to any one of items 28 to 33, wherein said anti-inflammatory agent is a steroid.
35. The use or composition for use according to item 34, wherein said steroid is a corticosteroid.
36. The use or composition for use according to item 35, wherein said corticosteroid is budenoside.
37. The use or composition for use according to any one of items 28 to 36, wherein the sinus administration is sinus rinse or irrigation.
38. The use or composition for use according to any one of items 28 to 37, wherein said composition comprises a mixture of different probiotic strains.
39. The use or composition for use according to any one of items 28 to 38, wherein said composition comprises a single probiotic strain.
40. The use or composition for use according to any one of items 28 to 39, wherein said probiotics comprise Gram-positive bacteria.
41. The use or composition for use according to item 40, wherein said Gram-positive bacteria are lactic acid bacteria (LAB).
42. The use or composition for use according to item 41, wherein said LAB is of the Lactococcus genus.
43. The use or composition for use according to item 42, wherein said LAB of the Lactococcus genus is Lactococcus lactis.
44. The use or composition for use according to item 43, wherein said Lactococcus lactis is Lactococcus lactis subsp. cremoris.
45. The use or composition for use according to any one of items 28 to 44, wherein the composition comprising probiotics is for twice-daily administration.
46. The use or composition for use according to any one of items 28 to 45, wherein the composition comprising probiotics is for administration for a period of at least two weeks.
47. The use or composition for use according to item 46, wherein the composition comprising probiotics is for administration for a period of about 3 to 5 weeks.
48. The use or composition for use according to any one of items 28 to 47, wherein the probiotics are present in an amount corresponding to at least about 10⁶ colony-forming units (CFU) in said composition.
49. The use or composition for use according to item 48, wherein the probiotics are present in an amount corresponding to about 10⁸ to about 10¹² CFU in said composition.
50. The use or composition for use according to any one of items 28 to 50, wherein the composition comprising probiotics is a liquid composition and comprises a concentration of probiotics of at least about 10⁵ CFU/mL.
51. The use or composition for use according to item 50, wherein the concentration of probiotics is about 10⁶ to about 10⁸ CFU/mL.
52. The use or composition for use according to item 50 or 51, wherein the liquid composition comprises a saline buffer.
53. The use or composition for use according to item 52, wherein said method further comprises resuspending said probiotics in said saline buffer prior to said use.
54. The use or composition for use according to any one of items 28 to 53, wherein said probiotics and said anti-inflammatory agent are in different compositions.
55. The use or composition for use according to any one of items 28 to 54, wherein said use improves at least one of the following CRS symptom parameters in said subject: sino-nasal symptom score (SNSS), Sino-nasal Outcome test 22 items (SNOT-22) score and/or peri-operative sinus endoscopy (POSE) score.
56. The use or composition for use according to item 55, wherein said use improves all of the following CRS symptom parameters in said subject: SNSS, SNOT-22 score, and POSE score.
57. The use or composition for use according to any one of items 28 to 56, wherein said steroid-resistant CRS is steroid-resistant CRS with nasal polyps.
58. The use or composition for use according to any one of items 28 to 57, wherein said steroid-resistant CRS is refractory CRS.
59. A kit or package for treating steroid-resistant CRS in a patient, for improving or restoring the response to anti-inflammatory treatment in a steroid-resistant CRS patient, said kit or package comprising a composition comprising an effective amount of probiotics and instructions setting forth the method of any one of items 1 to 27.
60. The kit or package of item 59, wherein the composition comprising probiotics is lyophilized, spray-dried or freeze-dried.
61. The kit or package of item 60, further comprising a solution for reconstituting said composition comprising probiotics.
62. The kit or package of any one of items 59 to 61, further comprising an anti-inflammatory agent.
63. The kit or package of any one of items 59 to 62, further comprising a device or system for sinus rinse or sinus irrigation.
64. The kit or package of any one of items 59 to 62, comprising one of more of the features defined in any one of items 3 to 27.

Other objects, advantages and features of the present invention will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1 shows the treatment protocol of the study described herein. Study was performed at one site (CHUM Hôtel-Dieu, Montreal): washout period for 14 days with only nasal and sinus irrigation with isotonic saline; 14 days of probiotic treatment followed by 14 days with only nasal and sinus irrigation with isotonic saline (corticosteroid therapy was reinitiated at that point), and finally a follow-up visit about 1 month after corticosteroid therapy re-initiation (1M or D60). D=days; M=months, conv.=conventional; 16S=swab culture for 16S bacteria sequencing.

FIGS. 2A to 2D show the effect of probiotic treatment on nasal and sinus symptom scores. FIG. 2A: Mean of total nasal and sinus symptom scores (TNSS) from SNSS questionnaire for all enrolled patients (n=24). FIG. 2B: effect of probiotic treatment on nasal congestion score. FIG. 2C: effect of probiotic treatment on the need to blow nose score. FIG. 2D: effect of probiotic treatment on post-nasal drip score. Response to treatment was determined by calculating time-weighted average scores over the study period from day 0 (D0) to day 60 (D60, 1 month after re-initiation of corticosteroid therapy).

FIGS. 3A to 3J show the effect of probiotic treatment on scores of a disease-specific quality of life (QOL) assessment instrument, the 22-item Sino-Nasal Outcome Test (SNOT-22). Higher score indicates greater impairment of QOL. Response to treatment is calculated from period D-14 to D60. (D-14=Day −14, 14 days before introduction of probiotic, interruption of corticosteroid therapy; D60=Day 60, 1 month after re-initiation of corticosteroid therapy). FIG. 3A: Mean of total score of the 22 items from quality of life (QOL) questionnaire for all enrolled patients (n=24). FIG. 3B shows the SNOT-22 scores for each category, namely rhinologic symptoms, extranasal rhinologic symptoms, ear/facial symptoms, psychological symptoms, and sleep dysfunction. FIG. 3C shows the SNOT scores for the rhinologic symptoms. FIG. 3D shows the SNOT scores for the “nasal obstruction” symptoms. FIG. 3E shows the SNOT scores for the “need to blow nose” symptoms. FIG. 3F shows the SNOT scores for the “runny nose” symptoms. FIG. 3G shows the SNOT scores for the “sneezing” symptoms. FIG. 3H shows the SNOT scores for the “thick nasal discharge” symptoms.

FIGS. 4A-4G show the effect of probiotic treatment on sinus endoscopy. Values represent mean score for all enrolled patients (n=24). Response to treatment is calculated from period D-14 to D60. FIG. 4A: Total POSE score; FIG. 4B: mucosal oedema score (ethmoid cavity); FIG. 4C: polypoid change score (ethmoid cavity); FIG. 4D: polyposis score; FIG. 4E: secretion score (ethmoid cavity); FIG. 4F: total score for ethmoid cavity; FIG. 4G: Frontal recess score. Values represent mean score for all enrolled patients (n=24).

FIG. 5 shows the impact of probiotic treatment on smell. UPSIT-40 scores prior to (D0; Day 0, left bars) and immediately following treatment (D14, Day 14, right bars) presented individually for all patients (n=24).

FIG. 6 shows boxplots of Shannon diversity indices before (D0; Day 0) and immediately following probiotic treatment (D14, Day 14).

FIG. 7 shows the Individual bacterial species statistical analysis. Before (D0, upper bars) vs. after (D14, lower bars) treatment conditions were compared in a two-group statistical test (White's non-parametric t-test, p-value <0.05, effect size >1.00, confidence interval at 95%). (Control=D0, Treatment=D14.).

FIGS. 8A and 8B show the effect of probiotic treatment on total symptoms in CRS patients without (CRSsNP, FIG. 8A) or with polyps (CRSwNP, FIG. 8B).

FIG. 9 shows the SNOT-22 scores in CRSsNP (left bars) and CRSwNP (right bars) patients.

FIGS. 10A and 10B show the POSE scores in CRSsNP (FIG. 10A) and CRSwNP (FIG. 10B) patients.

FIGS. 11A and 11B shows the POSE scores in the ethmoid cavity in CRSsNP (FIG. 11A) and CRSwNP (FIG. 11B) patients.

FIGS. 12A and 12B shows the POSE scores in secondary sinuses in CRSsNP (FIG. 12A) and CRSwNP (FIG. 12B) patients.

### DISCLOSURE OF INVENTION

The use of the terms “a” and “an” and “the” and similar referents in the context of describing the invention (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

All methods described herein can be performed in any suitable order unless otherwise indicated herein or otherwise clearly contradicted by context.

The use of any and all examples, or exemplary language (e.g., “such as”) provided herein, is intended merely to better illustrate the invention and does not pose a limitation on the scope of the invention unless otherwise claimed.

No language in the specification should be construed as indicating any non-claimed element as essential to the practice of the invention.

Herein, the term “about” has its ordinary meaning. The term “about” is used to indicate that a value includes an inherent variation of error for the device or the method being employed to determine the value, or encompass values close to the recited values, for example within 10% or 5% of the recited values (or range of values).

Unless otherwise defined, all technical and scientific terms used herein have the same meaning as commonly understood by one of ordinary skill in the art to which this invention belongs.

List of abbreviations used herein: Eos: Eosinophils; CFU: Colony-forming units; CI: Confidence interval; CRS: Chronic rhinosinusitis; CRSsNP: Chronic rhinosinusitis without nasal polyps; CRSwNP: Chronic rhinosinusitis with nasal polyps; ESS: Endoscopic sinus surgery; ET: Eustachian tube; IgE: Immunoglobulin E; L. lactis W136: Lactococcus lactis W136; MCID: Minimal clinically important difference; POSE: Peri-operative sinus endoscopy; QOL: Quality of life; SNSS: Sino-nasal symptom score; SNOT-22: Sino-nasal Outcome test 22 items; UPSIT-40: University of Pennsylvania Smell Identification Test 40-items; WBC: White blood cells.

In the studies described herein, the present inventors show that topical application of probiotics directly to the sinus cavities via irrigation is safe and effective in improving nasal symptoms, QOL indices, and mucosal aspect in a population of patients with refractory CRS. Probiotic therapy led to improvement in the subject's sinus conditions overall, as assessed by sinus symptoms, disease-specific QOL indices, and endoscopically-assessed mucosal aspect of the sinuses during the treatment period and a 14-day follow up observation period. Furthermore, whereas the symptoms of these patients could not be improved by corticosteroid therapy prior to probiotic treatment, re-initiation of corticosteroid therapy after probiotic treatment led to further improvements of sinus conditions and QOL parameters, which was more pronounced in patients with polyps, providing evidence that probiotic therapy improved (strengthened) or restored the ability of patients previously refractory to conventional treatments (including steroid treatment) to respond to corticosteroid therapy.

Accordingly, in a first aspect, the present invention provides a method of treating a patient suffering from steroid-resistant chronic rhinosinusitis (CRS), said method comprising nasally administering to said subject a composition comprising an effective amount of probiotics comprising Lactococcus lactis. The present invention also provides the use of a composition comprising an effective amount of probiotics comprising Lactococcus lactis for treating a patient suffering from steroid-resistant CRS, wherein said composition is for nasal administration. The present invention also provides the use of a composition comprising an effective amount of probiotics comprising Lactococcus lactis for the manufacture of a medicament for treating a patient suffering from steroid-resistant CRS, wherein said composition is for nasal administration. The present invention also provides a composition for nasal administration comprising an effective amount of probiotics comprising Lactococcus lactis for use in the treatment of a patient suffering from steroid-resistant CRS.

In another aspect, the present invention provides a method of treating a patient suffering from medical treatment-resistant (e.g., steroid-resistant) CRS, said method comprising nasally administering to said subject (i) a composition comprising an effective amount of probiotics; and (ii) an effective amount of an anti-inflammatory, e.g., a corticosteroid. The present invention also provides the use of (i) a composition comprising an effective amount of probiotics; and (ii) an effective amount of an anti-inflammatory, e.g., a corticosteroid, for treating a patient suffering from medical treatment-resistant (e.g., steroid-resistant) CRS. The present invention also provides the use of (i) a composition comprising an effective amount of probiotics; and (ii) an effective amount of an anti-inflammatory, e.g., a corticosteroid, for the manufacture of a medicament for treating a patient suffering from medical treatment-resistant (e.g., steroid-resistant) CRS. The present invention also provides (i) a composition for nasal administration comprising an effective amount of probiotics; and (ii) an effective amount of an anti-inflammatory, e.g., a corticosteroid, for use in the treatment of medical treatment-resistant (e.g., steroid-resistant) CRS.

In another aspect, the present invention provides a method of improving or restoring the response to anti-inflammatory (e.g., corticosteroid) nasal treatment in a patient suffering from medical treatment-resistant (e.g., steroid-resistant) CRS, said method comprising nasally administering to said subject a composition comprising an effective amount of probiotics. The present invention also provides the use of a composition comprising an effective amount of probiotics for improving or restoring the response to anti-inflammatory (e.g., corticosteroid) treatment in a patient suffering from medical treatment-resistant (e.g., steroid-resistant) CRS. The present invention also provides the use of a composition comprising an effective amount of probiotics for the manufacture of a medicament for improving or restoring the response to anti-inflammatory (e.g., corticosteroid) treatment in a patient suffering from steroid-resistant CRS. The present invention also provides a composition for nasal administration comprising an effective amount of probiotics for improving or restoring the response to anti-inflammatory (e.g., corticosteroid) treatment in a patient suffering from medical treatment-resistant (e.g., steroid-resistant) CRS.

In another aspect, the present invention provides a method of treating a patient suffering from chronic rhinosinusitis with nasal polyps (CRSwNP), said method comprising nasally administering to said subject a composition comprising an effective amount of probiotics comprising Lactococcus lactis. The present invention also provides the use of a composition comprising an effective amount of probiotics comprising Lactococcus lactis for treating a patient suffering from CRSwNP, wherein said composition is for nasal administration. The present invention also provides the use of a composition comprising an effective amount of probiotics comprising Lactococcus lactis for the manufacture of a medicament for treating a patient suffering from CRSwNP, wherein said composition is for nasal administration. The present invention also provides a composition for nasal administration comprising an effective amount of probiotics comprising Lactococcus lactis for use in the treatment of a patient suffering from CRSwNP.

In another aspect, the present invention provides a method of treating a patient suffering from CRSwNP, said method comprising nasally administering to said subject (i) a composition comprising an effective amount of probiotics; and (ii) an effective amount of an anti-inflammatory, e.g., a corticosteroid. The present invention also provides the use of (i) a composition comprising an effective amount of probiotics; and (ii) an effective amount of an anti-inflammatory, e.g., a corticosteroid, for treating a patient suffering from CRSwNP. The present invention also provides the use of (i) a composition comprising an effective amount of probiotics; and (ii) an effective amount of an anti-inflammatory, e.g., a corticosteroid, for the manufacture of a medicament for treating a patient suffering from CRSwNP. The present invention also provides (i) a composition for nasal administration comprising an effective amount of probiotics; and (ii) an effective amount of an anti-inflammatory, e.g., a corticosteroid, for use in the treatment of CRSwNP.

In another aspect, the present invention provides a method of improving or restoring the response to anti-inflammatory (e.g., corticosteroid) nasal treatment in a patient suffering from CRSwNP, said method comprising nasally administering to said subject a composition comprising an effective amount of probiotics. The present invention also provides the use of a composition comprising an effective amount of probiotics for improving or restoring the response to anti-inflammatory (e.g., corticosteroid) treatment in a patient suffering from CRSwNP. The present invention also provides the use of a composition comprising an effective amount of probiotics for the manufacture of a medicament for improving or restoring the response to anti-inflammatory (e.g., corticosteroid) treatment in a patient suffering from CRSwNP. The present invention also provides a composition for nasal administration comprising an effective amount of probiotics for improving or restoring the response to anti-inflammatory (e.g., corticosteroid) treatment in a patient suffering from CRSwNP.

The term “patient suffering from medical treatment-resistant (e.g., steroid-resistant) resistant CRS” as used herein refers to a patient whose condition, as assessed by conventional CRS symptom parameters such as those described herein, is not significantly improved by conventional medical treatment (e.g., sinus rinse, antibiotics and/or anti-inflammatory agents), and more particularly to medical treatment comprising steroids.

In an embodiment, the patient had not undergone sinus surgery prior to said treatment. In another embodiment, the patient had undergone sinus surgery prior to said treatment.

In an embodiment, the patient suffering from steroid-resistant CRS is a patient suffering from refractory CRS. The expression “patients suffering from refractory CRS” as used herein refers to patients with CRS refractory to medical and surgical therapy as defined by failure of technically adequate surgery followed by irrigations with a steroid (e.g., a corticosteroid such as budesonide) solution^{1,22} to eliminate the signs and symptoms of CRS. In an embodiment, the patient is non-responsive to a treatment with an anti-inflammatory agent (e.g., steroid), i.e. in whom cessation of anti-inflammatory agent (e.g., steroid) treatment results in no significant deterioration of the signs and/or symptoms of CRS. In another embodiment, the patient is responsive to a treatment with an anti-inflammatory agent (e.g., steroid), i.e. in whom cessation of anti-inflammatory agent (e.g., steroid) treatment results in deterioration of signs and/or symptoms of CRS (i.e., one or more of the symptoms and/or conditions described below). In an embodiment, the subject suffers from refractory CRS with nasal polyps.

As used herein, the term “treat” or “treatment” is not intended to be absolute terms. Treatment can refer to any delay in onset, reduction in the frequency or severity of symptoms, amelioration of one or more symptoms, improvement in patient comfort and/or respiratory function, etc. The effect of treatment can be compared to an individual or pool of individuals not receiving a given treatment, or to the same patient prior to, or after cessation of, treatment.

In an embodiment, the treatment comprises improvement of at least one sinus conditions. In another embodiment, the treatment comprises improvement of at least one quality-of-life (QOL) parameters. In another embodiment, the treatment comprises improvement of sinus mucosal aspect. In an embodiment, the treatment comprises improvement of at least one sinus conditions, at least one QOL parameters, and sinus mucosal aspect. In another embodiment, the treatment comprises improvement of at least one of the following CRS symptom parameters: sino-nasal symptom score (SNSS), 22 item Sino-Nasal Outcome Test (SNOT-22®, Washington University School of Medicine, St. Louis, Mo.) score and/or peri-operative sinus endoscopy (POSE) score. In a further embodiment, the treatment comprises improvement of at least two of the above-noted CRS symptom parameters. In a further embodiment, the treatment comprises improvement of all of the above-noted CRS symptom parameters. In another embodiment, the treatment improves (decreases) the SNOT-22 score by at least 9.

The SNSS comprises five items (facial pain, headache, nasal congestion, need to blow nose and post-nasal drip). In an embodiment, the treatment comprises improvement of at least one of the SNSS items. In an embodiment, the treatment comprises improvement of at least two of the SNSS items. In an embodiment, the treatment comprises improvement of at least three of the SNSS items. In an embodiment, the treatment comprises improvement of at least four of the SNSS items. In an embodiment, the treatment comprises improvement of all five of the SNSS items. In an embodiment, the treatment comprises improvement (reduction) of facial pain. In an embodiment, the treatment comprises improvement (reduction) of nasal congestion. In an embodiment, the treatment comprises improvement (reduction) of need to blow nose. In an embodiment, the treatment comprises improvement (reduction) of post-nasal drip. In a further embodiment, the treatment comprises improvement of facial pain, nasal congestion, need to blow nose and post-nasal drip.

The SNOT-22 comprises 22 items including 12 nasal- and sinus-related symptoms (need to blow nose, nasal blockage, sneezing, runny nose, cough, post-nasal discharge, thick nasal discharge, ear fullness, dizziness, ear pain, facial pain/pressure, decreased sense of smell/taste) and 10 psychological and behavioral symptoms (difficulty falling asleep, wake up at night, lack of a good night's sleep, wake up tired, fatigue, reduced productivity, reduced concentration, frustrated/restless/irritable, sad and embarrassed) (Quintanilla-Dieck, et al., International Forum of Allergy & Rhinology 2012; 2(6):437-443). The items may also be subdivided into 5 domains/categories namely (1) rhinologic symptoms, (2) extranasal rhinologic symptoms, (3) ear/facial symptoms, (4) psychological dysfunctions, and (5) sleep dysfunctions. In an embodiment, the treatment comprises improvement of at least one rhinologic symptoms. In an embodiment, the treatment comprises improvement of at least one psychological dysfunctions. In an embodiment, the treatment comprises improvement of at least one sleep dysfunctions. In an embodiment, the treatment comprises improvement of at least one, two, three, four, five, six or seven of the SNOT-22 items. In an embodiment, the treatment comprises improvement (reduction) of at least one, two, three, four, five, six or seven of the following SNOT symptoms: “frustration/restless/irritable” symptoms, “nasal obstruction” symptoms, “need to blow nose” symptoms, “reduced productivity” symptoms, “runny nose” symptoms, “sneezing” symptoms and “thick nasal discharge” symptoms. In an embodiment, the treatment comprises improvement (reduction) of 1, 2, 3, 4 or 5 of the following SNOT symptoms: “nasal obstruction” symptoms, “need to blow nose” symptoms, “runny nose” symptoms, “sneezing” symptoms and “thick nasal discharge” symptoms.

The POSE score comprises the following items: (1) middle turbinate score (normal or synechia/lateralized), (2) middle meatus/MMA score (healthy, narrowing/closure and maxiliary sinus content); (3) ethmoid cavity score (healthy, crusting, mucosal edema, polypoid change, polyposis and secretions; and (4) secondary sinuses score (frontal recess/sinus and sphenoid sinus. In an embodiment, the treatment comprises improvement of the total POSE score. In an embodiment, the treatment comprises improvement of at least one, two or three of the above-noted scores (1)-(4). In an embodiment, the treatment comprises improvement of ethmoid cavity score. In an embodiment, the treatment comprises improvement of middle meatus/MMA score. In an embodiment, the treatment comprises improvement of secondary sinuses score. In an embodiment, the treatment comprises improvement of ethmoid cavity, middle meatus/MMA and secondary sinuses scores. In an embodiment, the treatment comprises improvement of ethmoid cavity and secondary sinuses scores.

In an embodiment, the treatment comprises improvement of at least one of (one, two, three, four or five) the total POSE score, the polypoid change score (ethmoid cavity), the secretion score (ethmoid cavity), the total score for ethmoid cavity, and the frontal recess score. In an embodiment, the treatment comprises improvement of the total POSE score. In an embodiment, the treatment comprises improvement of the polypoid change score (ethmoid cavity). In an embodiment, the treatment comprises improvement of the secretion score (ethmoid cavity). In an embodiment, the treatment comprises improvement of the total score for ethmoid cavity. In an embodiment, the treatment comprises improvement of the frontal recess score. In an embodiment, the treatment comprises improvement of the total POSE score, the polypoid change score (ethmoid cavity), the secretion score (ethmoid cavity), the total score for ethmoid cavity, and the frontal recess score.

The treatment may also be assessed using other methods such as improvement of the 10-symptom visual analog score (VAS) (Ryan, et al., Laryngoscope 2011; 121:674-678), computed tomography (CT) (e.g., an improved Lund-Mackay score) (Hopkins et al., Otolaryngology—Head and Neck Surgery 2007; 137:555-561), and/or endoscopy score (an improved Lund-Kennedy score).

The term “improving or restoring the response to anti-inflammatory (e.g., corticosteroid) treatment” means that the response (e.g., in terms of reduction of one or more symptoms of CRS) to an anti-inflammatory agent (e.g., corticosteroid) after the administration of the composition comprising an effective amount of probiotics is better or higher relative to the response prior to administration of the composition comprising an effective amount of probiotics.

The term “effective amount” refers to that amount of a therapeutic agent sufficient to ameliorate refractory CRS, e.g., to ameliorate one or more symptoms of refractory CRS. For example, for a given disease parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

The term “probiotics” as used herein refers to microorganisms that are generally recognized as safe (GRAS) and that confers a health benefit on the host when administered in a suitable amount. Strains of the following genera are known to be probiotics: Lactobacillus, Bifidobacterium, Saccharomyces (e.g., Saccharomyces boulardii), Enterococcus (e.g., Enterococcus durans, Enterococcus faecium), Streptococcus (e.g., Streptococcus thermophilus), Pediococcus (e.g., Pediococcus acidilactici), Leuconostoc (e.g., Leuconostoc mesenteroides), Lactococcus (e.g., Lactococcus lactis), Bacillus (e.g., Bacillus coagulans, Bacillus subtilis, Bacillus cereus), Escherichia coli (e.g., Escherichia coli Nissle 1917) and the like (see, e.g., Fijan, Int J Environ Res Public Health. 2014 May; 11(5): 4745-4767).

Specific examples of probiotic bacterial species that may be included in the probiotic compositions include, but are not limited to: Bacillus capillosus, Bacillus coagulans, Bacillus lentils, Bacillus licheniformis, Bacillus mesentericus, Bacillus natio, Bacillus pumilus, Bacillus subtilis; Bacteroides amylophilus, Bacteroides ruminocola, Bacteroides suis; Bifidobacterium adolescends, Bifidobacterium animalis, Bifidobacterium bifidum, Bifidobacterium breve, Bifidobacterium infantis, Bifidobacterium lactis, Bifidobacterium longum, Bifidobacterium pseudolongum, Bifidobacterium thermophilum; Enterococcus cremoris, Enterococcus diacetylactis, Enterococcus faechim, Enterococcus intermedins, Enterococcus lactis, Enterococcus muntdi, Enterococcus thermophilic; Escherichia coli, Lactobacillus acidophilus, Lactobacillus alimentarius, Lactobacillus amvlovorus, Lactobacillus brevis, L. hulgaricus, Lactobacillus casei, Lactobacillus cellobiosus, Lactobacillus crispatus, Lactobacillus curvatus, Lactobacillus delbrueckii, Lactobacillus delbrueckii ss. hulgaricus, Lactobacillus farciminis, Lactobacillus fermentum, Lactobacillus gasseri, Lactobacillus helveticus, Lactobacillus jensenii, Lactobacillus johnsonii, Lactobacillus lactis, Lactobacillus lactis subsp. lactis, Lactobacillus lactis subsp. cremoris, Lactobacillus paracasei, Lactobacillus plantarum, Lactobacillus plantarum strain LM, Lactobacillus reuteri, Lactobacillus rhamnosus, Lactobacillus sakei, Lactobacillus salivarius; Leuconostoc mesenteroides; Pediococcus acidilactici; Pediococcus pentosaceus; Propionihacterium freudenreichii, Propionibacterium shermanii; Staphylococcus carnosiis, Staphylococcus xylosus; Streptococcus infantarius, Streptococcus salivarius ss. thermophilus, Streptococcus thermophilus, and Streptococcus lactis.

In an embodiment, the probiotics comprise Gram-positive bacteria. Representative Gram-positive bacteria include strain of the Lactococcus, Lactobacillus and Bifidobacterium genus. The genus Lactobacillus includes various Gram-positive facultative anaerobic or microaerophilic rod-shaped bacteria, and are a major part of the lactic acid bacteria (LAB) group (including Lactobacillus, Lactococcus, Enterococcus, Oenococcus, Pediococcus, Streptococcus and Leuconostoc species) that can convert hexose sugars to lactic acid thus producing an acid environment.

Examples of probiotic Lactobacillus spp. strains that may be used in the methods described herein include Lactobacillus rhamnosus, Lactobacillus acidophilus, Lactobacillus reuteri, Lactobacillus casei, Lactobacillus paracasei, Lactobacillus plantarum, Lactococcus lactis, Lactococcus delbrueckii Lactobacillus reuteri, Lactobacillus salivahus, Lactobacillus helveticus, Lactobacillus parabuchneri, Lactobacillus johnsonii, Lactobacillus pentosus, Lactobacillus brevis, Lactobacillus sakei, Lactobacillus diolivorans, Lactobacillus buchneri, Lactobacillus gasseri, Lactobacillus fermentum and Lactobacillus bulgaricus.

Examples of Bifidobacterium spp. strains that may be used in the methods described herein include Bifidobacterium bifidum, Bifidobacterium longum, Bifidobacterium breve, Bifidobacterium pseudolongum, Bifidobacterium infantis, Bifidobacterium adolescentis, Bifidobacterium animalis (subsp. Lactis) and Bifidobacterium lactis.

In an embodiment, the composition comprises a mixture of different probiotic strains, i.e. a mixture of two, three, four, five or more different probiotic strains (e.g., bacterial strains). In another embodiment, the composition comprises a single probiotic strain (e.g., a single bacterial strain).

In an embodiment, the probiotics comprise lactic acid Gram-positive bacteria. In a further embodiment, the lactic acid Gram-positive bacteria is of the Lactococcus genus. In a further embodiment, the lactic acid Gram-positive bacteria is Lactococcus lactis, preferably Lactococcus lactis subsp. cremoris (also sometimes referred to as Streptococcus lactis subsp. cremoris), which are commonly used in food fermentation (notably fermented milk products). Examples of Lactococcus lactis subsp. cremoris stains include L. lactis subsp. cremoris SK11, MG1363, IL1403, 2250, 9B4, W136, A17, A76, AM2, AM4, AM5, ATCC 19257, CM1-3, CNCM 1-1631, DCH-4, DN030087 (CNCM 1-2807), GE214, IMN-C18, LMG 2130, MG1614, ML1, MSUA2, NCDO 763/ML3, NCC2287 (CNCM 1-4157), NCK436, NIAI H-61, NIRD HC-1, NIRD Ho-6, NIZO B78, NZ9800, P8-2-47, UC503, UC509.9, UC653, US3, W34, Wg2 and NIZO B1157 (see, e.g., UniProt Taxonomy, mnemonic organism identification code LACLC, NCBI Taxon identifier 1359).

Probiotic bacteria for inclusion in the compositions of the present invention may be derived from a variety of different sources including commercial sources, including but not limited to international depositories (e.g., America Type Culture Collection—ATCC®) and other national equivalents (e.g., Collection Nationale De Cultures De Microorganismes—CNC; China General Microbiological Culture Collection Center—CGMCC, and the like).

In some embodiments, the sinus administration/delivery is performed using an inhalation device, a spray, an aerosol, a syringe, nasal drops, nebulization, atomization, by flushing, or by irrigation. In an embodiment, the administration is performed by sinus rinse or irrigation, e.g., irrigation to the nasal and sinus passages. Sinus delivery/administration may be achieved/performed using commercially available kits or systems, e.g., NeilMed SINUS RINSE™ system, hydraSense NetiRinse® Nasal & Sinus Irrigation Kit, SinuPulse™ Elite Advanced Nasal Sinus Irrigation System, Xlear™ Sinus Care Rinse system, Optinose®, or Navage® irrigator devices.

In an embodiment, the composition comprising probiotics may be administered at any suitable frequency, for example three times a day, twice-daily, daily, once every two days, twice a week, once a week, every ten days or once per two weeks. In an embodiment, the composition comprising probiotics is administered twice-daily.

The composition comprising probiotics defined herein may be administered for a period suitable to achieve beneficial effects, for example at least one week, at least 10 days, at least 2 weeks, at least 3 weeks, at least 4 weeks, at least 6 weeks, at least 9 weeks, or at least 12 weeks. In embodiments, the composition comprising probiotics defined herein is administered for a period of about 2 weeks to about 6, 7 or 8 weeks, for example for a period of about 3-5 weeks. In an embodiment, the composition comprising probiotics defined herein is administered for about 1 month (28 days or 4 weeks). In a further embodiment, the composition comprising probiotics is administered twice-daily for about 1 month (28 days). The composition comprising probiotics defined herein can be administered during several periods, preferably with a rest period between two periods of treatments. In embodiments, two or more rounds of administration of the composition comprising probiotics defined herein may be performed, for example a first round of administration is performed (e.g., over 2, 3, 4 or more weeks), followed by an interruption (e.g., for 1, 2, 3, 4 or more weeks), and followed by a second round of administration. The composition comprising probiotics may be the same (e.g., same probiotics or combination of probiotics in the same amount) or different in each round of administration.

In embodiment, the probiotics are present in an amount corresponding to at least about 10⁵ colony-forming units (CFU), at least about 10⁶ CFU, at least about 10⁷ CFU, at least about 10⁸ CFU, or at least about 10⁹ CFU in said composition. In further embodiments, the probiotics are present in an amount of about 10⁵, 10⁶, 10⁷ or 10⁸ CFU to about 10⁹, 10¹⁰, 10¹¹ or 10¹² CFU, for example about 10⁸ CFU to about 10¹⁰ CFU, e.g., about 10⁹ CFU in said composition. The term “colony forming units” (“CFU”) is defined as number of bacterial cells as revealed by microbiological counts on agar plates.

In an embodiment, the composition is a liquid composition (solution or suspension) and comprises a concentration of probiotics of at least about 10⁴ CFU/mL, at least about 10⁵ CFU/mL, at least about 10⁶ CFU/mL, at least about 10⁷ CFU/mL, or at least about 10⁸ CFU/mL. In further embodiments, the composition comprises a concentration of probiotics of about 10⁴ CFU/mL, 10⁵ CFU/mL, or 10⁶ CFU/mL to about 10⁸, 10⁹, 10¹⁰ or 10¹¹ CFU/mL, for example about 10⁶ CFU/mL to about 10⁸ CFU/mL, for example about 10⁷ CFU/mL.

In an embodiment, the anti-inflammatory agent is a corticosteroid. The corticosteroids commonly used for the treatment of CRS include beclomethasone dipropionate, triamcinolone acetonide, flunisolide, budesonide, prednisolone, fluticasone propionate, mometasone furoate, ciclesonide, and fluticasone furoate. In an embodiment, the corticosteroid is administered to a sinus, i.e. is in a composition for sinus administration.

In an embodiment, the above-mentioned methods or uses further comprise the administration or use of other active agents or treatments for CRS, including antibiotics such as macrolides (e.g., erythromycin, azithromycin); penicillins (e.g., amoxicillin, beta-lactam, ampicillin); tetracyclines (e.g., doxycycline, tetracycline); sulfonamides (e.g., mafenide, sulfacetamide); fluoroquinolones; cephalosporins (e.g., ceftaroline fosamil, ceftobiprole), polymyxin B, other anti-inflammatory agents, leukotriene inhibitors (e.g., montelukast), saline washes, etc. In a further embodiment, the above-mentioned methods or uses further comprise the administration or use of a macrolide antibiotic, such as azithromycin.

In an embodiment, the compositions defined herein further comprise one or more pharmaceutically acceptable carriers or excipients, for example an aqueous carrier or excipient. A variety of aqueous carriers can be used, e.g., buffered saline. These solutions are generally free of undesirable matter, e.g., contaminating species. The compositions can contain pharmaceutically acceptable auxiliary substances to approximate/reproduce physiological conditions such as pH adjusting and buffering agents, for example, sodium acetate, sodium chloride, potassium chloride, calcium chloride, sodium lactate, etc.

As used herein, the term “pharmaceutically acceptable” is used synonymously with physiologically acceptable and pharmacologically acceptable with respect to, e.g., a pharmaceutical composition. A pharmaceutical composition will generally comprise agents for buffering and preservation in storage, and can include buffers and carriers for appropriate delivery. Therapeutic formulations are prepared using standard methods known in the art by mixing the active ingredient(s) with one or more optional pharmaceutically acceptable carriers, excipients and/or stabilizers (see, e.g., Remington: The Science and Practice of Pharmacy, by Loyd V Allen, Jr, 2012, 22^{nd} edition, Pharmaceutical Press; Handbook of Pharmaceutical Excipients, by Rowe et al., 2012, 7^{th} edition, Pharmaceutical Press). The composition defined herein may be prepared in any suitable form which does not negatively affect the viability of the probiotics forming the composition. Selection of the excipients and the most appropriate methods for formulation in view of the particular purpose of the composition is within the scope of ordinary persons skilled in the art of pharmaceutical technology. In an embodiment, the composition comprising probiotics defined herein is freeze-dried, spray-dried or lyophilized, is in a powder form (see, e.g., Fonseca et al., Methods Mol Biol. 2015; 1257:477-88). The composition may optionally comprise one or more ingredients for improving viability during storage (protectants), such as low molecular weight sugars (e.g., sorbitol, mannitol, trehalose, sucrose), cell components, etc. Such freeze-dried, spray-dried or lyophilized composition may be reconstituted into a liquid composition using a suitable solution, for example a saline solution. The probiotics present in the composition may be encapsulated, e.g., in a polymer or gel matrix.

In an embodiment, the compositions defined herein further comprise one or more excipients commonly used in nasal formulations (e.g., nasal sprays), such as water, preservatives (e.g., EDTA, benzalkonium chloride, benzethonium chloride, benzyl alcohol, chlorobutanol, methylparaben, phenylethyl alcohol, phenylmercuric acetate, propylene paraben, thimerosal), buffer salts or tonicity agents (e.g., sodium chloride, potassium chloride, glycerol, glycine), viscosity modifying agents (e.g., Me-OH—Pr cellulose, Na-CMC, microcrystalline cellulose), surfactants (glyceryl monoleate, lecithin, polysorbate 20 or 80) suspending agents/solvents (PEG), pH adjusting agents (acetic and citric acids, NaOH, HCl), mucoadhesive polymers (e.g., HPMC, chitosan), fillers/binders (e.g., cellulose-based polymers, maltodextrin, propylene glycol, colloidal silicon dioxide, guar gum) and flavors (e.g., menthol, saccharin sodium, sorbitol).

In an embodiment, the composition comprising probiotics defined herein and the corticosteroid are co-administered (e.g., consecutively, simultaneously, at different times). In an embodiment, the co-administration is coextensive, i.e. occurs during overlapping periods of time. In another embodiment, the composition comprising probiotics defined herein and the corticosteroid are administered consecutively, and in a further embodiment the composition comprising probiotics is administered prior to administration of the corticosteroid. In another embodiment, the corticosteroid is administered before and after the administration of the composition comprising probiotics defined herein. In another embodiment, the corticosteroid is administered before, during and after the administration of the composition comprising probiotics defined herein.

In another embodiment, the anti-inflammatory agent (e.g., corticosteroid) is present in the composition comprising the probiotics. Accordingly, in another aspect, the present invention provides a composition comprising an effective amount of probiotics and an anti-inflammatory agent (e.g., corticosteroid). In another aspect, the present invention provides a composition for treating a patient suffering from refractory CRS, said composition comprising an effective amount of probiotics and an anti-inflammatory agent (e.g., corticosteroid). In an embodiment, the composition further comprises other agents useful for the management of CRS, e.g., antibiotics, other anti-inflammatory agents, saline washes, etc.

In an embodiment, the probiotics and the anti-inflammatory agent are in solid form, for example a dry powder. Probiotics and anti-inflammatory agents in dry powder form may be administered with a suitable device/system for dry powder nasal drug delivery (dry powder inhalers), for example the powder nasal delivery devices from Teijin® or Optinose®, Turbuhaler®, Diskus®, Diskhaler®, Clickhaler® or Ultrahaler®.

In another embodiment, the corticosteroid and the probiotics are in different compositions, but these compositions are present together in the same kit or package. In an embodiment, the composition comprising probiotics is lyophilized, spray-dried or freeze-dried. The kit or package can optionally include one or more containers, saline solutions (e.g., to reconstitute the lyophilized, spray-dried or freeze-dried compositions prior to administration), buffers, written instructions, reference to an internet site, or electronic instructions (e.g. on a support such as a CD-ROM, DVD, USB flash drive), and more particularly instructions for performing the methods and uses described herein, i.e. for treating refractory CRS in a patient, for improving or restoring the response to anti-inflammatory (e.g., corticosteroid) treatment in a refractory CRS patient, etc. In some embodiments, the kit or package of the invention will include a case for holding the reagents (compositions comprising probiotics, anti-inflammatory agent, saline solution, etc.) in the kit or package, which can be included separately or in combination. In an embodiment, the kit or package comprises a lyophilized, spray-dried or freeze-dried composition comprising probiotics and a (saline) solution for reconstituting the lyophilized, spray-dried or freeze-dried compositions. In an embodiment, the lyophilized, spray-dried or freeze-dried composition comprising probiotics is in a first container and the (saline) solution is in a second container. In an embodiment, the kit or package further comprises an anti-inflammatory agent, e.g., a corticosteroid. In a further embodiment, the anti-inflammatory agent is in a third container. In another embodiment, the kit or package further comprises a device or system for delivering the composition(s) to the subject's nasal passage and sinuses, including the systems for sinus rinse or sinus irrigation, or the powder nasal delivery devices, described above.

The subject/patient to be treated may be a child, an adult or an elderly. In an embodiment, the subject/patient is an adult or an elderly. In another embodiment, the subject/patient also suffers from asthma.

Any and all combinations and subcombinations of the embodiments and features disclosed herein are encompassed by the present invention. For example, in an embodiment, the composition defined herein comprises about 10⁸ to about 10¹⁰ Gram-positive bacteria, for example Lactococcus lactis bacteria (e.g., Lactococcus lactis subsp. cremoris). In another example, the composition comprising probiotics is administered once- or twice-daily for a period of about 4 to 6 weeks, in combination with budesonide.

### MODE(S) FOR CARRYING OUT THE INVENTION

The present invention is illustrated in further details by the following non-limiting examples.

### Example 1: Materials and Methods

Study Design

The trial took place at the Centre Hospitalier de l'Université de Montreal (CHUM), an established tertiary academic rhinology reference center, and was performed by a single physician specialized in ENT (Martin Desrosiers). The probiotic used was a Lactococcus lactis subsp. cremoris strain (L. lactis strain W136) furnished by AGROPUR (St. Hubert, QC, Canada). Approvals from Health Canada and the CHUM Institutional Review Board and Ethics committee approval were obtained prior to trial performance.

Twenty-four patients with chronic rhinosinusitis refractory to medical and surgical therapy as defined by failure of technically adequate surgery followed by irrigations with budesonide solution^{1,22} were enrolled in a prospective, open-label trial. Excluded were patients <18 years, cystic fibrosis, with technical reasons for ESS failure, active sinus infection with purulence, pain and/or hyperthermia, or with immune suppression from disease or medication.

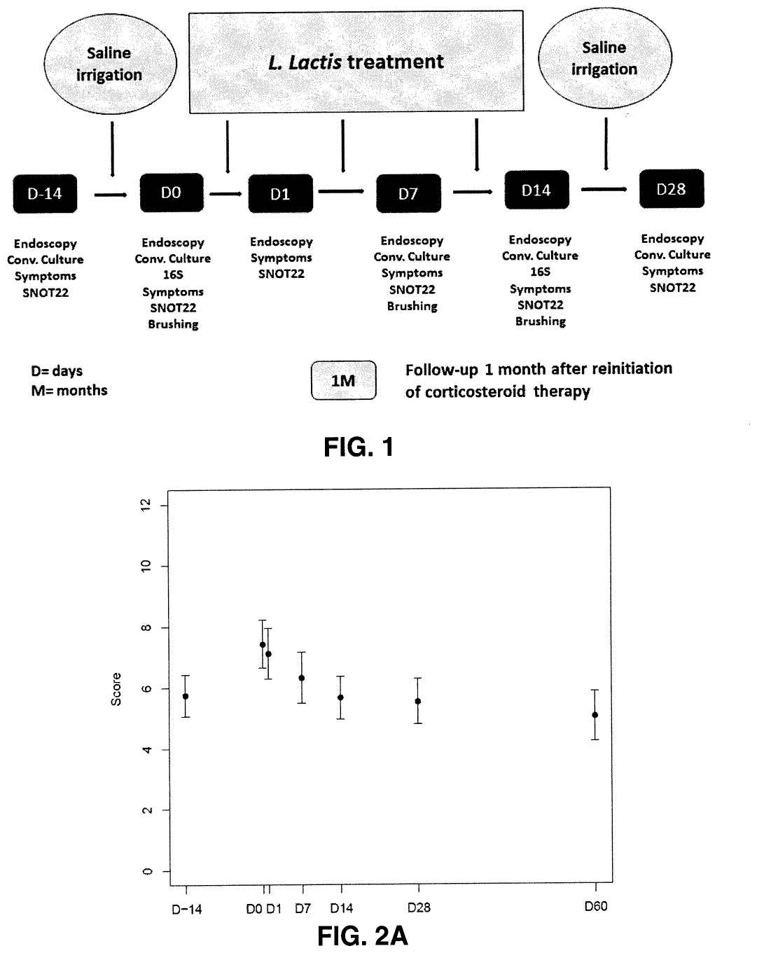

The treatment protocol is outlined in FIG. 1. Following recruitment, a two-week (14-day) washout period occurred during which all sinus medications were ceased, with the exception of nasal and sinus irrigation with 120 mL of 0.9% saline solution administered twice-daily using the NeilMed™ Pediatric Sinus Rinse system (NeilMed Pharmaceuticals Inc., Santa Rosa, Calif.).

This was followed by the treatment period where patients were initially treated with 14 days of BID irrigations containing the reconstituted live probiotic L. lactis W136 at 1.2×10⁹ CFU (120 mL; 10⁷ CFU/mL) concentration. This was followed by a 14-day period during where only irrigation with isotonic saline was administered, and then corticosteroid treatment (irrigation with budesonide solution) was resumed. Measures were obtained at the beginning and end of the washout period, at the initiation of probiotic therapy, and one, seven, fourteen and twenty-eight days after initiation of therapy, as well as one month after probiotic therapy.

Frozen L. lactis W136 for reconstitution was provided to participants in single-dose containers in an insulated transport box for transfer to their home freezer. This was reconstituted in 120 mL of buffered saline 0.9% solution using a pediatric size NeilMed™ SinusRinse bottle. A new irrigation bottle was supplied for each application.

Patients were assessed for symptomatology by questionnaire (Sinonasal symptom score (SNSS), disease-specific quality of life (Sino-nasal Outcome Test 22 item (SNOT-22) questionnaire and aspect of the sinus mucosa as assessed by endoscopy (POSE score).^{24,25} Safety was assessed prospectively for: i) sense of smell using the UPSIT-40²⁶ (40-item University of Pennsylvania Smell Identification Test), ii) potential impact on middle ear and Eustachian tube function by otomicroscopy, iii) endoscopically-obtained swab cultures (COPAN, Becton & Dickinson Company, Sparks, Md., USA) to identify overgrowth by pathogenic or probiotic bacteria.

Microbiome Analysis

Exploratory analysis of the microbiome to identify changes in the sinus microbiome occurring during probiotic therapy was performed using 16S rRNA sequencing. Endoscopically-obtained swabs of the frontal recess were collected at the initiation and immediately after probiotic administration (D0, D14) for each patient. 16S rRNA extraction, amplification, and sequencing (Illumina® MiSeq Reagent Kit v2; Illumina, San Diego, Calif.) was performed at the Farnsworth Family Genomic facility at McMaster University.

Culture-independent 16S microbiome analysis was performed by Dr. Emmanuel Gonzalez, a bioinformatician from the Canadian Centre for Computational Genomics based at the McGill University and Genome Quebec Innovation Centre (Montreal, QC, Canada). Raw sequencing data underwent bioinformatic analysis, including quality control (adapter clipping, read trimming based on phred score, read discarding, chimera detection), 16S taxonomic assignments, diversity measurements (alpha and beta diversity) and multi-variate biostatistical analysis. Results are presented as i) estimate of richness using Shannon diversity index ii) standardized abundance overall community chart, and iii) individual species.

Statistical Analysis

Primary endpoint was improvement in symptom score, with secondary endpoints of improvement in QOL and mucosal score as assessed by sinus endoscopy. Response to treatment was determined by calculating time-weighted average scores over the study period, using Time=Day 0 or D-14 as the baseline value. Results were expressed in term of confidence intervals (CI).

### Example 2: Results—Demographics

Twenty-seven patients who met inclusion/exclusion criteria were recruited. One patient was withdrawn before initiation of the trial because of scheduling issues, and two other patients withdrawn during the saline treatment period as their symptoms became intolerable following withdrawal of usual sinus medication. None of these three patients received any probiotic therapy.

24 patients received sinus probiotic therapy and all successfully completed the study. Their baseline characteristics are summarized in Table 1. All had undergone previous ESS, 17/24 for an initial diagnosis of CRSwNP. None were current smokers, 21% reported respiratory allergies, and 75% were under treatment for asthma.

TABLE 1
Population baseline characteristics. (n =
Number; SD = Standard Deviation)

| | |
|---|---|
| CRSwNP/CRSsNP (n) | 17/7 |
| Age (mean ± SD) | 54.9 ± 11.9 years |
| Gender (n) | Female = 13 (54%) |
| Ethnicity (n) | 22 (Caucasian); 2 (Arabic) |
| Asthma (n (%)) | 18 (75%) |
| Number of previous ESS (mean ± SD) | 2.08 ± 1.14 |
| History of Allergy (Seasonal) (n (%)) | 5 (21%) |
| Current smoker | 0 (0%) |
| WBC (10⁹/L) (mean ± SD) | 6.83 ± 1.29 |
| Eos (10⁹/L) (mean ± SD) | 0.39 ± 0.32 |
| IgE kIU/L(mean ± SD) | 275.8 ± 397.7 |

### Example 3: Results—Efficacy

Sino-Nasal Symptom Score (SNSS):

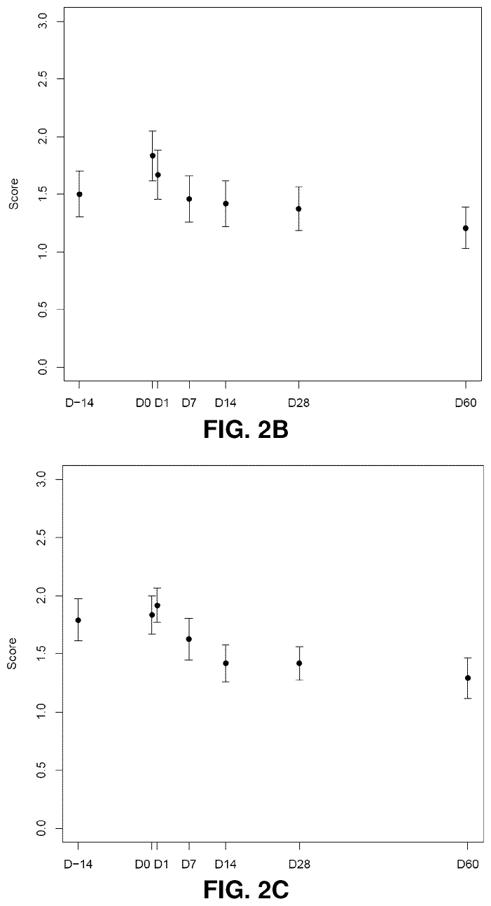

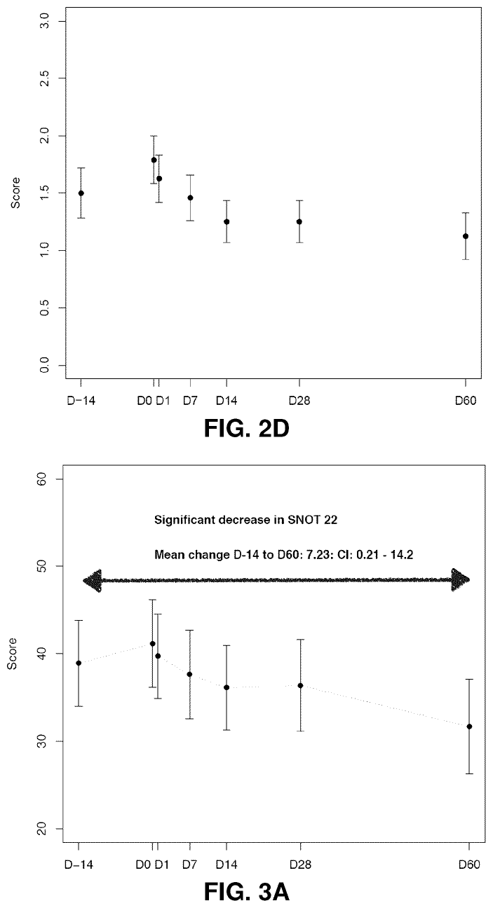

The aggregate symptom score and individual scores showed favorable response to probiotic therapy (FIGS. 2A-2D). Total symptom scores initially deteriorated during the saline-only washout period, then improved significantly over the 28-day treatment and observation period (Mean=6.0; 95% CI: 4.65 to 7.36). Pattern of response showed score improving during the 14 days of probiotic administration, with improvements maintained over the 14-day post-treatment observation period. Further reduction of the scores was seen for several symptoms after re-initiation of corticosteroid therapy (D60). Individual symptoms of nasal congestion (FIG. 2B), need to blow nose (FIG. 2C) and post-nasal drip (FIG. 2D) all followed a similar pattern of improvement. Symptoms showing greatest magnitudes of improvement were for the symptoms of “Nasal congestion” (95% CI: 1.10 to 1.81), “Post nasal drip” (95% CI: 1.04 to 1.67) and “Need to blow nose” (95% CI: 1.26 to 1.81). Importantly, further improvements of sinus symptoms were observed one month after corticosteroid therapy (irrigation with budesonide solution) was resumed (D60). For example, a significant improvement of the “Need to blow nose” symptom was observed (FIG. 2C, 95% CI: −0.93 to −0.07) between D-14 (time at which corticosteroid therapy was stopped) and D60 (1 month after corticosteroid therapy was resumed), thus providing evidence that probiotic therapy improved or restored the ability of these patients to respond to corticosteroid therapy.

Disease-Specific Quality of Life (QOL) Measures.

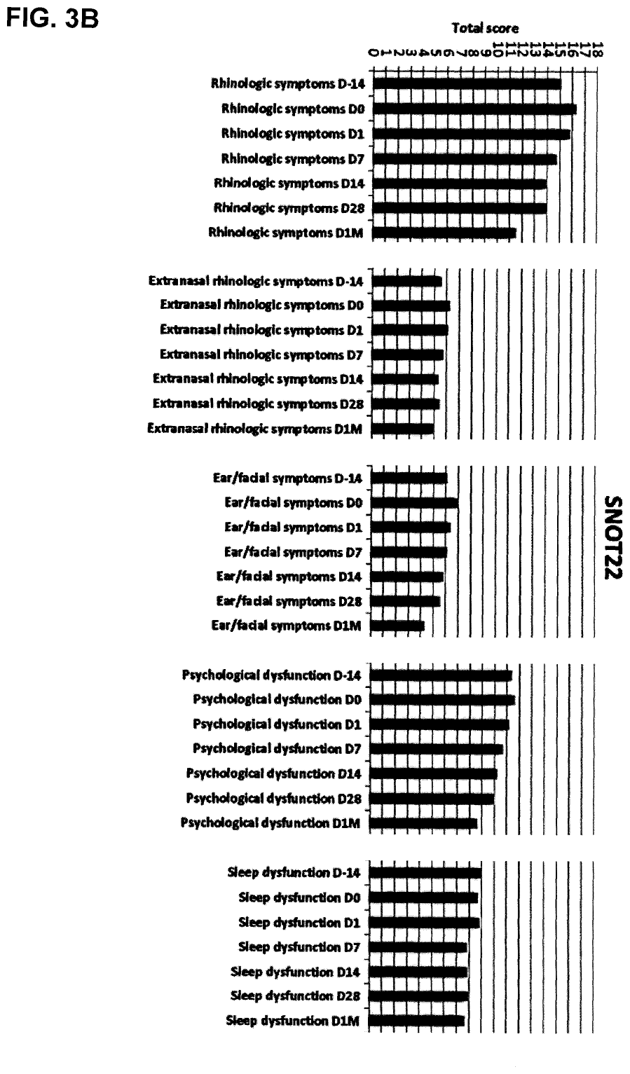

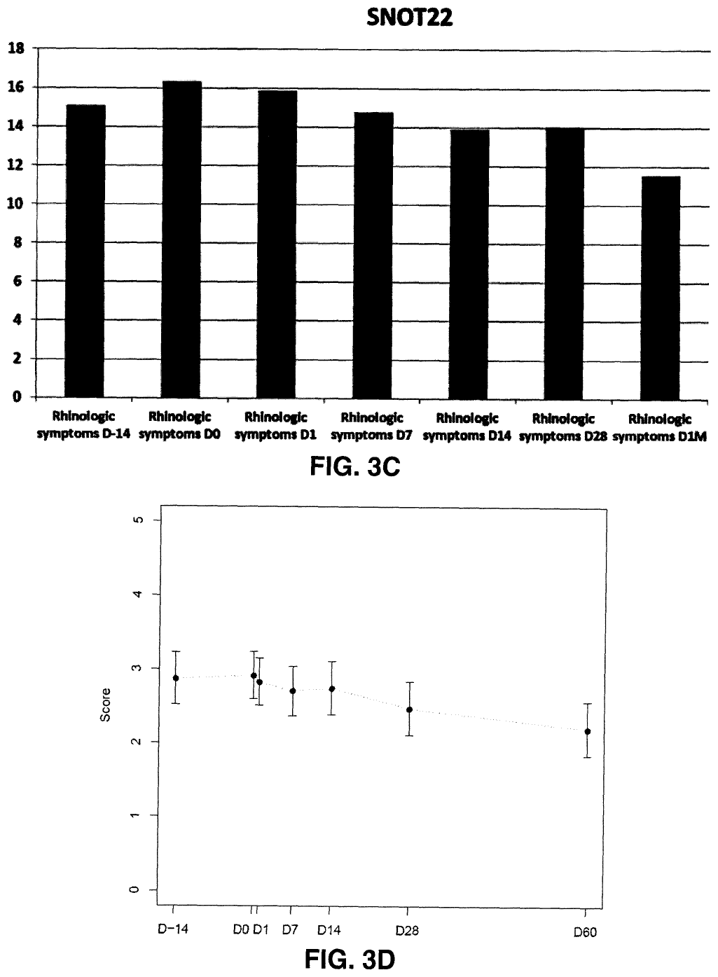

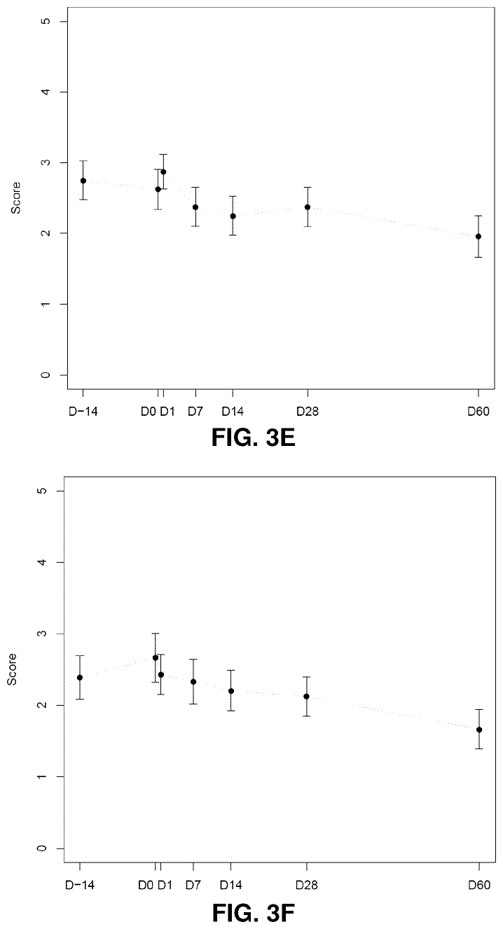

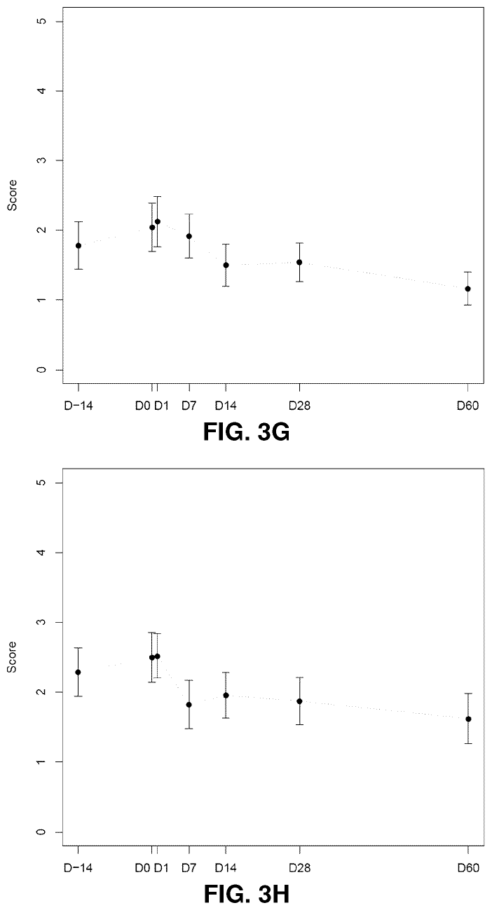

SNOT-22 score followed a similar pattern as for symptomatology. SNOT-22 score deteriorated following withdrawal of pre-study sinus medication and then significantly improved over the course of the trial (95% CI: 27.28 to 46.87). Again, improvement persisted following administration of probiotic, and was maintained at the 28-day point, and further reductions of the SNOT-22 were observed 1 month after re-initiation of corticosteroid therapy (FIG. 3A-3H). A significant improvement was observed for the total SNOT-22 score between D-14 and D60 (FIG. 3A). Rhinologic symptoms showed the most significant improvements (FIGS. 3C-3H).

Endoscopic Score.

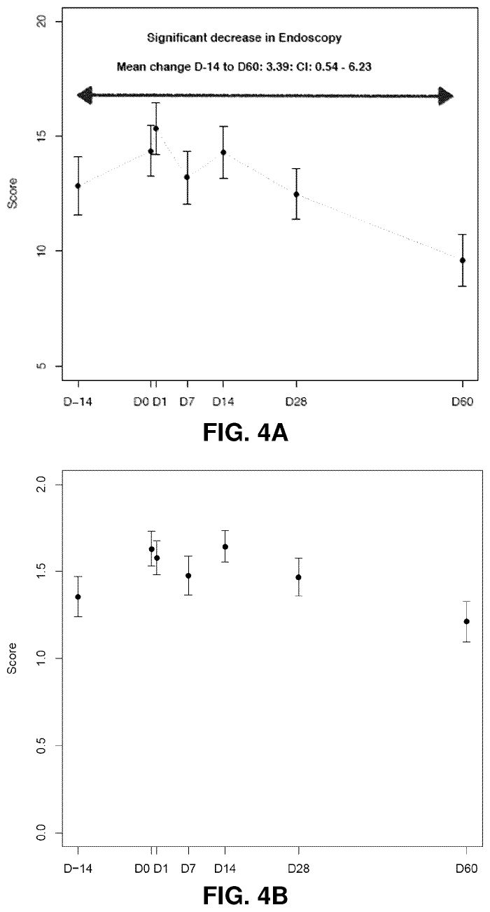

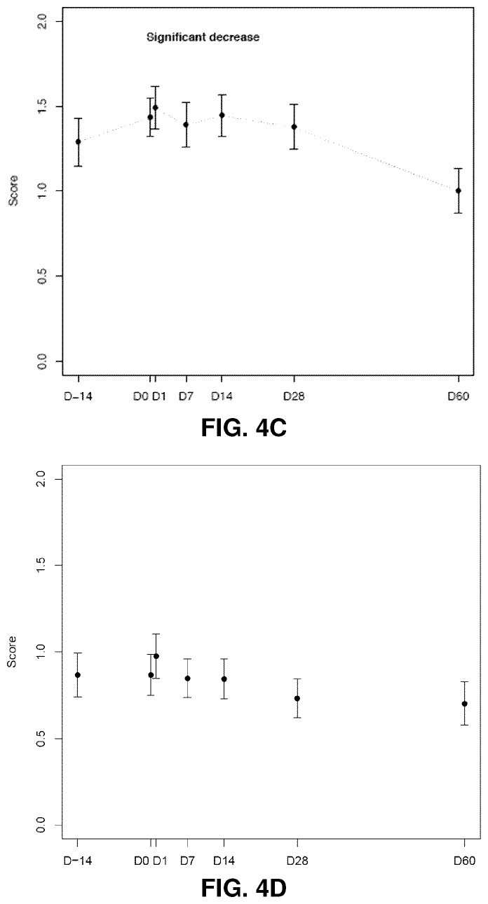

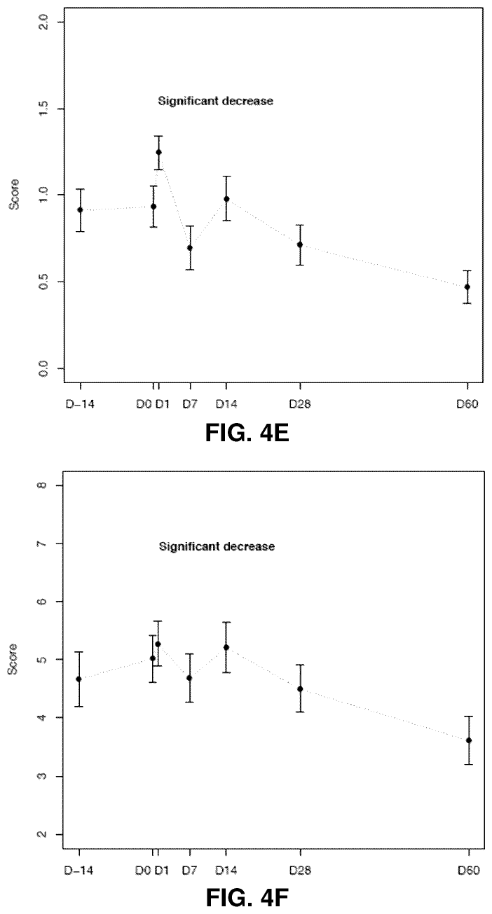

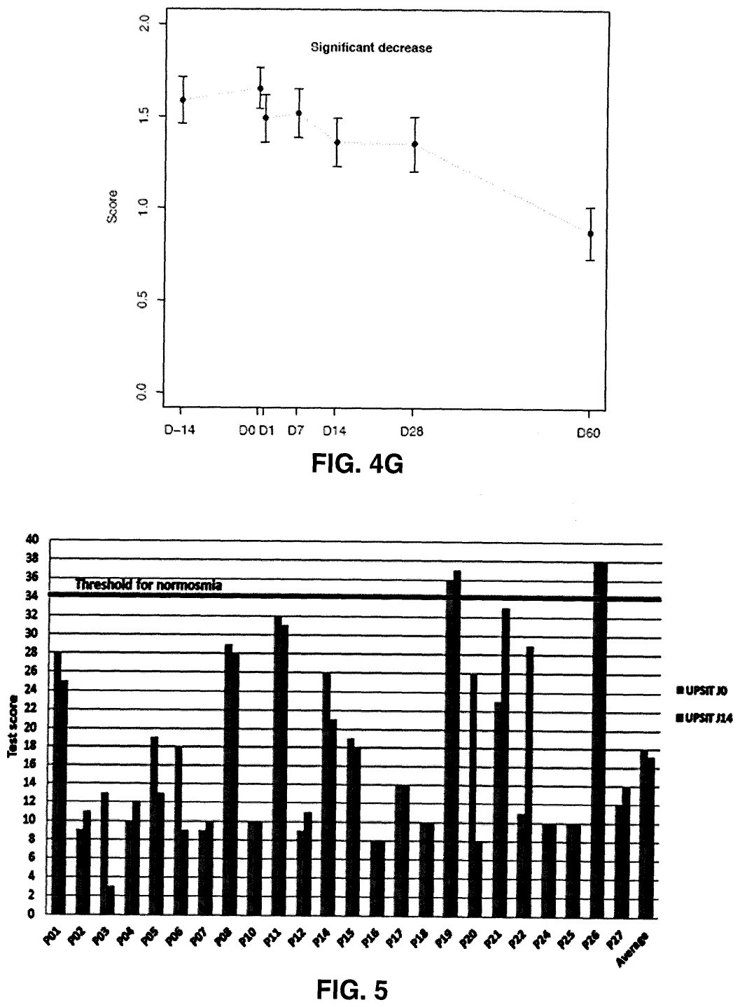

Endoscopic scores showed a similar pattern of improvement as for sinus symptoms and QOL between D0 and D28 (total score: 95% CI: 10.32 to 16.28) (FIGS. 4A-4G), again with further improvements observed after corticosteroid therapy was resumed (D60). A significant improvement was observed for the total POSE score (FIG. 4A), the polypoid change score (ethmoid cavity) (FIG. 4C), the secretion score (ethmoid cavity) (FIG. 4E), the total score for ethmoid cavity (FIG. 4F), and the frontal recess score (FIG. 4G) between D-14 and D60. This is consistent with the results above and provides evidence that probiotic therapy improved or restored the ability of the patients to respond to corticosteroid therapy.

### Example 4: Results—Safety

No serious adverse events occurred during the clinical trial.

Conventional Bacteriology:

No noticeable change in patterns of collection or in type of bacteria collected was observed over the course of treatment. There was no overgrowth of new pathogenic species and no growth of L. lactis on conventional culture before or after administration of the probiotic.

Olfactory Testing:

UPSIT-40 scores remained stable during the trial, with no evidence of impairment from therapy (95% CI: −3.46 to 2.13) (FIG. 5).

Middle Ear Status:

Two patients showed evidence of middle ear effusion during the trial period, without symptoms or signs of acute infection. One patient had effusion and otorrhea at inclusion. All patients reported ear fullness and ear pain in pre-treatment SNOT-22, suggesting pre-existing Eustachian tube (ET)/middle ear disease. Effusion resolved completely in all patients without additional treatment at one month follow-up after end of study.

Patient Self-Reported Events:

Patient self-reported minor events occurring during the trial but not related to the probiotic treatment were: Headache, migraines, nasal congestion, dental infection, throat pain, cold sore, gastroenteritis, nasal allergy episode, and shoulder pain.

### Example 5: Results—Microbiome Analysis

Exploratory 16S Microbiome Analysis:

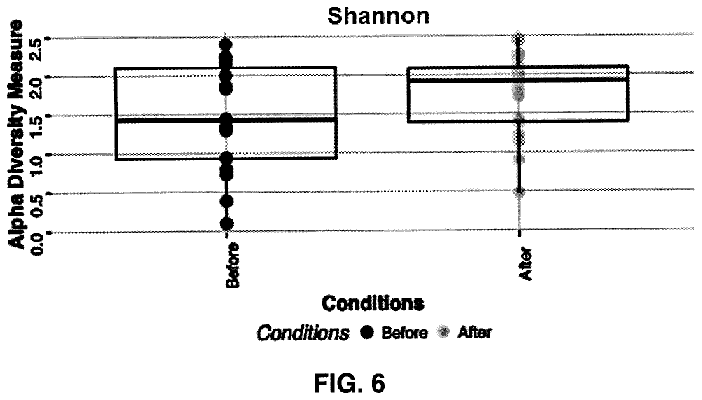

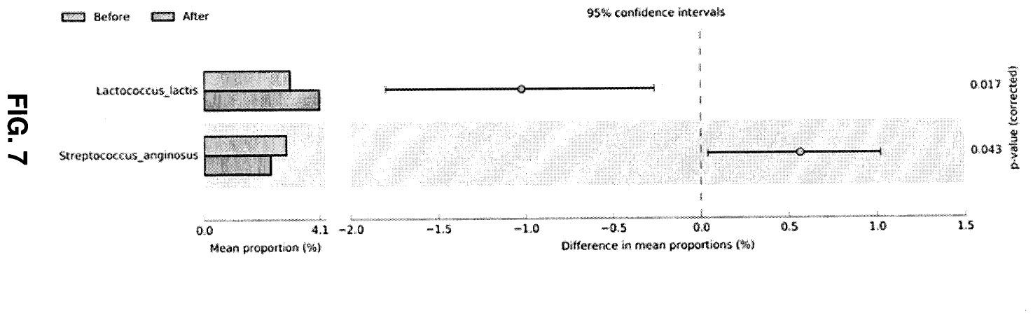

Bacterial diversity improved with treatment, as shown by an increase of the mean Shannon indices following probiotic administration (FIG. 7). For individual species (FIG. 6), significant increases in Lactococcus lactis sp. were observed, as was a decrease in the presence of Streptococcus anginosus, which is an important cause of morbidity in the respiratory tract with the propensity to form abscesses and cause invasive pyogenic infection³². There was also a trend to reduction in Staphylococcus aureus and Pseudomonas aeruginosa following treatment.

### Example 6: Results—CRSsNP/CRSwNP Sub-Group Analysis

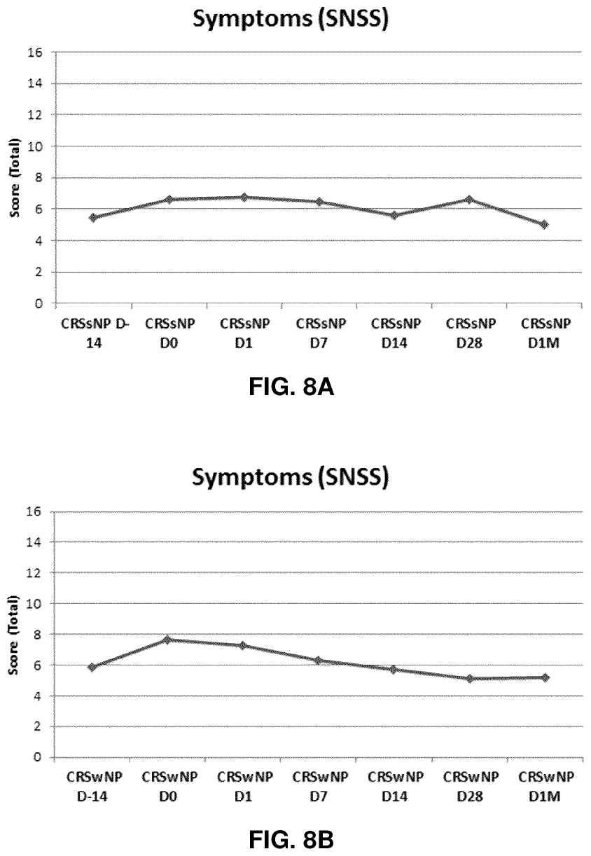

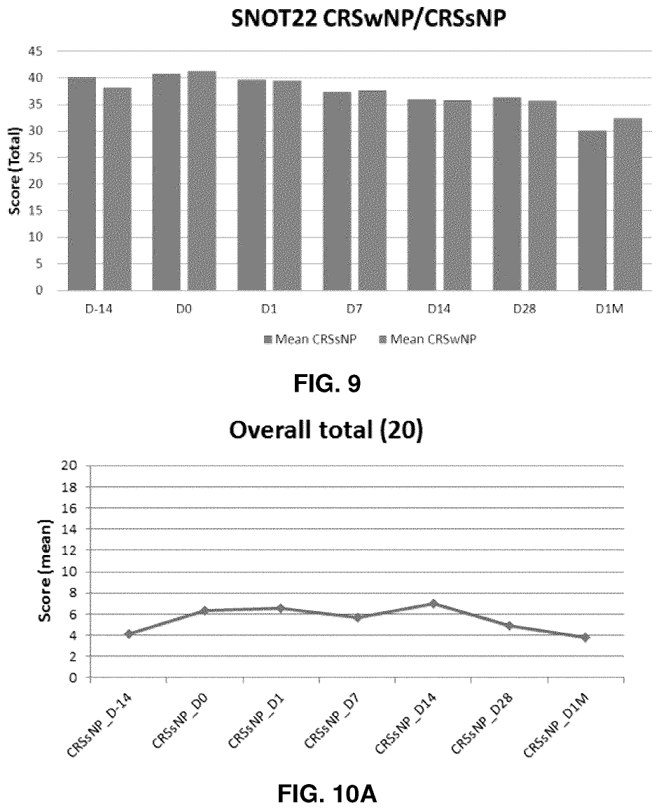

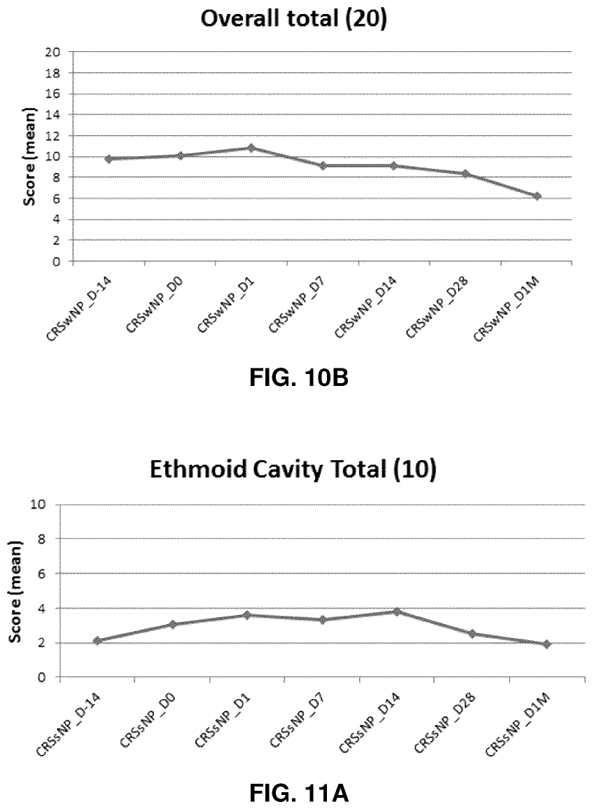

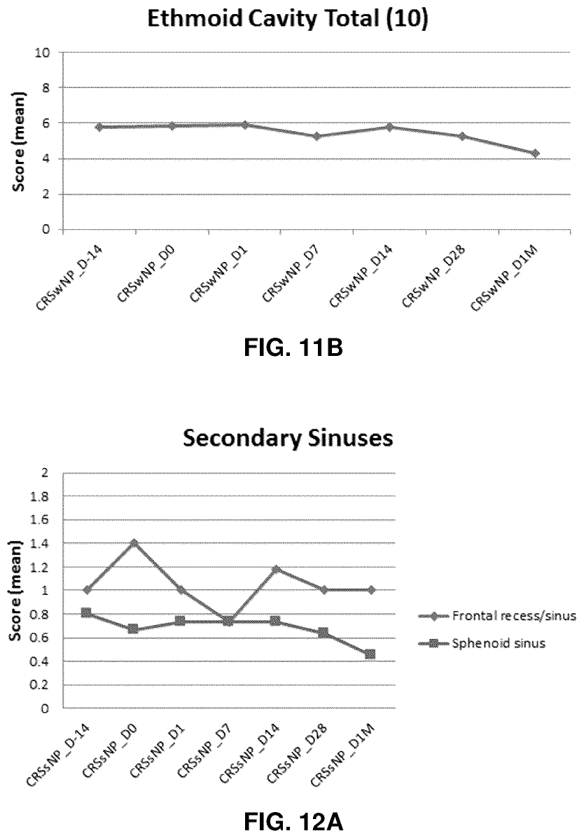

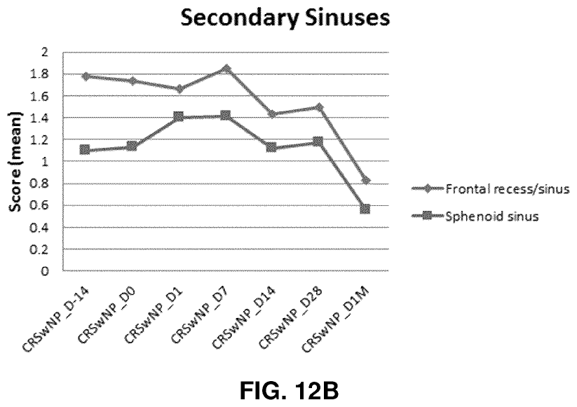

Sub-group analysis was performed according to clinical phenotype classification CRSsNP (n=7) and CRSwNP (n=17) (FIGS. 8 to 12). Of all assessed variables, the only significant difference was for endoscopic polyp score in CRSwNP compared to CRSsNP (Mean=0.91; 95% CI: 0.08 to 1.74) over the period D0 to D28. Further improvements of the certain parameters, notably the POSE score, were observed after corticosteroid therapy was resumed (as evidenced by the decrease between D28 and D1M), particularly in the CRSwNP group and in the secondary sinuses (FIG. 12B).

### CONCLUSION

The results presented herein show that topical application of probiotics directly to the sinus cavities via irrigation is safe and effective in improving symptoms, QOL indices, and mucosal aspect in a population of patients with refractory CRS. Probiotic therapy led to improvement in the subject's sinus conditions overall, as assessed by sinus symptoms, disease-specific quality of life indices, and endoscopically-assessed mucosal aspect of the sinuses during the treatment period and a 14-day follow up observation period. These effects in marked contrast to the increase in these parameters that had been observed following withdrawal of usual medication during the period which preceded probiotic administration where only irrigation with saline was received. Whereas some of these patients were non-responsive to corticosteroid therapy prior to probiotic treatment, re-initiation of corticosteroid therapy after probiotic treatment led to improvements of sinus conditions and QOL parameters, providing evidence that probiotic therapy improved or restored the ability of these patients to respond to corticosteroid therapy.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

### REFERENCES

1. Meltzer E O, Hamilos D L, Hadley J A, Lanza D C et al. Rhinosinusitis: Establishing definitions for clinical research and patient care. Otolaryngol Head Neck Surg. 2004; 131, S1-62.
2. Desrosiers M, Evans G A, Keith P K, Wright E D, Kaplan A, Bouchard J et al. Canadian clinical practice guidelines for acute and chronic rhinosinusitis. Allergy Asthma Clin Immunol. 2011; 7(1):2.
3. Hasten D, Fokkens W J, Bachert C et al. Chronic rhinosinusitis in Europe—an underestimated disease. A GA(2)LEN study. Allergy 2011; 66:1216-1223.
4. Anand V K. Epidemiology and economic impact of rhinosinusitis. The Annals of otology, rhinology & laryngology Supplement 2004; 193:3-5.
5. Rudmik L, Smith T L, Schlosser R J, Hwang P H, Mace J C, Soler Z M. Productivity costs in patients with refractory chronic rhinosinusitis. The Laryngoscope 2014; 124(9):2007-12.
6. Van Zele T, Claeys S, Gevaert P et al. Differentiation of chronic sinus diseases by measurement of inflammatory mediators. Allergy 2006; 61:1280-1289.
7. Stephenson M F, Mfuna L, Dowd S E et al. Molecular characterization of the polymicrobial flora in chronic rhinosinusitis. J Otolaryngol Head Neck Surg. 2010; 39:182-187.
8. Nader M-E, Abou-Jaoude P, Cabaluna M, Desrosiers M. Using response to a standardized treatment to identify phenotypes for genetic studies of chronic rhinosinusitis. J Otolaryngol Head Neck Surg. 2010; 39:69-75.
9. Stjarne P, Olsson P, Alenius M. Use of mometasone furoate to prevent polyp relapse after endoscopic sinus surgery. Arch Otolaryngol Head Neck Surg. 2009; 135:296-302.
10. Al-Shemari H, Abou-Hamad W, Libman M, Desrosiers M. Bacteriology of the sinus cavities of asymptomatic individuals after endoscopic sinus surgery. J Otolaryngol. 2007; 36:43-8.
11. Snelling A M. Effects of probiotics on the gastrointestinal tract. Curr Opin Infect Dis. 2005; 18(5):420-6.
12. Wolvers D, Antoine J M, Myllyluoma E, Schrezenmeir J, Szajewska H, Rijkers G T. Guidance for substantiating the evidence for beneficial effects of probiotics: prevention and management of infections by probiotics. J Nutr. 2010; 140(3):6985-712S.
13. Aroniadis O C, Brandt L J. Fecal microbiota transplantation: past, present and future. Curr Opin Gastroenterol. 2013; 29:79-84.
14. Abreu N A, Nagalingam N A, Song Y, Roediger F C, Pletcher S D, Goldberg A N, Lynch S V. Sinus microbiome diversity depletion and Coryne-bacterium tuberculostearicum enrichment mediates rhinosinusitis. Sci Transl Med. 2012; 4:151ra124.
15. Cleland E J, Drilling A, Bassiouni A, James C, Vreugde S, Wormald P J. Probiotic manipulation of the chronic rhinosinusitis microbiome. Int Forum Allergy Rhinol. 2014; 4(4):309-14.
16. Cope E K, Lynch S V. Novel microbiome-based therapeutics for chronic rhinosinusitis. Curr Allergy Asthma Rep. 2015; 15(3):504.
17. Tano K, Grahn Hakansson E, Holm S E, Hellstrom S. A nasal spray with alpha-haemolytic streptococci as long term prophylaxis against recurrent otitis media. Int J Pediatr Otorhinolaryngol. 2002; 62(1):17-23.
18. Skovbjerg S, Roos K, Holm S E, Grahn Hakansson E, Nowrouzian F, Ivarsson M, Adlerberth I, Wold A E. Spray bacteriotherapy decreases middle ear fluid in children with secretory otitis media. Arch Dis Child. 2009; 94:92-8.
19. Santagati M, Scillato M, Muscaridola N, Metoldo V, La Mantia I, Stefani S. Colonization, safety, and tolerability study of the Streptococcus salivarius 24SMBc nasal spray for its application in upper respiratory tract infections. Eur J Clin Microbiol Infect Dis. 2015; 34(10):2075-80.
20. Marchisio P, Santagati M, Scillato M, Baggi E, Fattizzo M, Rosazza C, Stefani S, Esposito S, Principi N. Streptococcus salivarius 24SMB administered by nasal spray for the prevention of acute otitis media in otitis-prone children. Eur J Clin Microbiol Infect Dis. 2015; 34(12):2377-83.
21. Mårtensson A, Greiff L, Lamei S S, Lindstedt M, Olofsson T C, Vasquez A, Cervin A. Effects of a honeybee lactic acid bacterial microbiome on human nasal symptoms, commensals, and biomarkers. Int Forum Allergy Rhinol. 2016; 6(9):956-63.
22. Desrosiers M, Evans G A, Keith P K, et al. Canadian clinical practice guidelines for acute and chronic rhinosinusitis. Allergy Asthma Clin Immunol. 2011; 7:2.
23. Schwartz J S, Peres A G, Mfuna Endam L, Cousineau B, Madrenas J, Desrosiers M. Topical probiotics as a therapeutic alternative for chronic rhinosinusitis: A preclinical proof of concept. Am J Rhinol Allergy. 2016; 30(6):202-205.
24. Hopkins C, Gillett S, Slack R, Lund V J, Browne J P. Psychometric validity of the 22-item Sinonasal Outcome Test. Clin Otolaryngol 2009; 34:447-454.
25. Wright E, Agrawal S. Impact of perioperative systemic steroids on surgical outcomes in patients with chronic rhinosinusitis with polyposis: evaluation with the novel Perioperative Sinus Endoscopy (POSE) scoring system. Laryngoscope 2007; 117(11 Pt 2 Suppl 115):1-28.
26. Doty R L, Shaman P, Dann M. Development of the University of Pennsylvania Smell Identification Test: a standardized microencapsulated test of olfactory function. Physiol Behav. 1984; 32(3): 489-502.
27. Oelschlaeger T A. Mechanisms of probiotic actions—A review. Int J Med Microbiol. 2010; 300(1):57-62.
28. De Moreno de Leblanc A, Del Carmen S, Zurita-Turk M, Santos Rocha C, van de Guchte M, Azevedo V, Miyoshi A, Leblanc J G. Importance of IL-10 modulation by probiotic microorganisms in gastrointestinal inflammatory diseases. ISRN Gastroenterol. 2011; 2011:892971.
29. Fischer K, Stein K, Ulmer A J, Lindner B, Heine H, Hoist O. Cytokine-inducing lipoteichoic acids of the allergy-protective bacterium Lactococcus lactis G121 do not activate via Toll-like receptor 2. Glycobiology 2011; 21(12):1588-1595.
30. Kaesler S, Skabytska Y, Chen K M, Kempf W E, Volz T, Koberle M, Wolbing F, Hein U, Hartung T, Kirschning C, Rocken M, Biedermann T. Staphylococcus aureus-derived lipoteichoic acid induces temporary T-cell paralysis independent of Toll-like receptor 2. J Allergy Clin Immunol. 2016; 138(3):780-790.e6.
31. Biedermann T, Skabytska Y, Kaesler S and Volz T. Regulation of T cell immunity in atopic dermatitis by microbes: the Yin and Yang of cutaneous inflammation. Front. Immunol. 2015; 6:353.
32. Sibley C D, Sibley K A, Leong T A, Grinwis M E, Parkins M D, Rabin H R, Surette, M G. The Streptococcus milleri Population of a Cystic Fibrosis Clinic Reveals Patient Specificity and Intraspecies Diversity. Journal of Clinical Microbiology 2010; 48(7):2592-2594.
33. Schleifer, K H, Kraus J, Dvorak C, Kilpper-Bälz R, Collins M D, and Fischer W. Transfer of Streptococcus lactis and related streptococci to the genus Lactococcus gen. nov. Syst. Appl. Microbiol. 1985; 6:183-195.
34. Stoikes N F, Dutton J M.), The effect of endoscopic sinus surgery on symptoms of Eustachian tube dysfunction. Am J Rhinol. 2005; 19(2):199-202.)
35. Anastasios Maniakas, Martin Desrosiers, Marc-Henri Asmar, Mohammad Al Falasi, Leandra Mfuna Endam, Claire Hopkins, et al. Eustachian tube symptoms are frequent in chronic rhinosinusitis and respond well to endoscopic sinus surgery. Otolaryngology Head and Neck Surgery 2017, in press.
36. Bachert C, Mannent L, Naclerio R M, Mullol J et al. Effect of Subcutaneous Dupilumab on Nasal Polyp Burden in Patients With Chronic Sinusitis and Nasal Polyposis: A Randomized Clinical Trial. JAMA. 2016; 315: 469-79.
37. Bachert C, Zhang L, Gevaert P. Current and future treatment options for adult chronic rhinosinusitis: Focus on nasal polyposis. J Allergy Clin Immunol. 2015; 136(6):1431-40.

What is claimed is:

1. A method of treating a patient suffering from steroid-resistant chronic rhinosinusitis (CRS), said method comprising administering to a sinus from said subject (i) a composition comprising an effective amount of probiotics; and (ii) an effective amount of an anti-inflammatory agent.

2. A method of improving or restoring the response to sinus treatment with an anti-inflammatory agent in a patient suffering from steroid-resistant chronic rhinosinusitis (CRS), said method comprising administering to a sinus from said subject a composition comprising an effective amount of probiotics.

3. The method of claim 1 or 2, wherein said anti-inflammatory agent is a steroid.

4. The method of claim 3, wherein said steroid is a corticosteroid.

5. The method of claim 4, wherein said corticosteroid is budenoside.

6. The method of any one of claims 1 to 5, wherein the administering is by sinus rinse or irrigation.

7. The method of any one of claims 1 to 6, wherein said composition comprises a mixture of different probiotic strains.

8. The method of any one of claims 1 to 6, wherein said composition comprises a single probiotic strain.

9. The method of any one of claims 1 to 8, wherein said probiotics comprise Gram-positive bacteria.

10. The method of claim 9, wherein said Gram-positive bacteria are lactic acid bacteria (LAB).

11. The method of claim 10, wherein said LAB is of the Lactococcus genus.

12. The method of claim 11, wherein said LAB of the Lactococcus genus is Lactococcus lactis,

13. The method of claim 12, wherein said Lactococcus lactis is Lactococcus lactis subsp. cremoris.

14. The method of any one of claims 1 to 13, wherein the composition comprising probiotics is administered twice-daily.

15. The method of any one of claims 1 to 14, wherein the composition comprising probiotics is administered for a period of at least two weeks.

16. The method of claim 15, wherein the composition comprising probiotics is administered for a period of about 3 to 5 weeks.

17. The method of any one of claims 1 to 16, wherein the probiotics are present in an amount corresponding to at least about 10⁶ colony-forming units (CFU) in said composition.

18. The method of claim 17, wherein the probiotics are present in an amount corresponding to about 10⁸ to about 10¹² CFU in said composition.

19. The method of any one of claims 1 to 18, wherein the composition comprising probiotics is a liquid composition and comprises a concentration of probiotics of at least about 10⁵ CFU/mL.

20. The method of claim 19, wherein the concentration of probiotics is about 10⁶ to about 10⁸ CFU/mL.

21. The method of claim 19 or 20, wherein the liquid composition comprises a saline buffer.

22. The method of claim 21, wherein said method further comprises resuspending said probiotics in said saline buffer prior to administration.

23. The method of any one of claims 1 to 22, wherein said probiotics and said anti-inflammatory agent are in different compositions.

24. The method of any one of claims 1 to 23, wherein said method improves at least one of the following CRS symptom parameters in said subject: sino-nasal symptom score (SNSS), Sino-nasal Outcome test 22 items (SNOT-22) score and/or peri-operative sinus endoscopy (POSE) score.

25. The method of claim 24, wherein said method improves all of the following CRS symptom parameters in said subject: SNSS, SNOT-22 score, and POSE score.

26. The method of any one of claims 1 to 25, wherein said steroid-resistant CRS is steroid-resistant CRS with nasal polyps.

27. The method of any one of claims 1 to 26, wherein said steroid-resistant CRS is refractory CRS.

28. Use of (i) a composition comprising an effective amount of probiotics; and (ii) an effective amount of an anti-inflammatory agent, for treating a patient suffering from steroid-resistant CRS, wherein said composition comprising an effective amount of probiotics and said anti-inflammatory agent are for sinus administration.

29. Use of (i) a composition comprising an effective amount of probiotics; and (ii) an effective amount of an anti-inflammatory agent, for the manufacture of a medicament for treating a patient suffering from steroid-resistant CRS, wherein said composition comprising an effective amount of probiotics and said anti-inflammatory agent are for sinus administration.

30. A combination for treating a patient suffering from steroid-resistant CRS, said combination comprising (i) a composition comprising an effective amount of probiotics; and (ii) an effective amount of an anti-inflammatory agent, wherein said composition comprising an effective amount of probiotics and said anti-inflammatory agent are for sinus administration.

31. Use of a composition comprising an effective amount of probiotics for improving or restoring the response to anti-inflammatory sinus treatment in a patient suffering from steroid-resistant CRS, wherein said composition is for sinus administration.

32. Use of a composition comprising an effective amount of probiotics for the manufacture of a medicament for improving or restoring the response to anti-inflammatory sinus treatment in a patient suffering from steroid-resistant CRS, wherein said composition is for sinus administration.

33. A composition comprising an effective amount of probiotics for improving or restoring the response to anti-inflammatory sinus treatment in a patient suffering from steroid-resistant CRS, wherein said composition is for sinus administration.

34. The use or composition for use according to any one of claims 28 to 33, wherein said anti-inflammatory agent is a steroid.

35. The use or composition for use according to claim 34, wherein said steroid is a corticosteroid.

36. The use or composition for use according to claim 35, wherein said corticosteroid is budenoside.

37. The use or composition for use according to any one of claims 28 to 36, wherein the sinus administration is sinus rinse or irrigation.

38. The use or composition for use according to any one of claims 28 to 37, wherein said composition comprises a mixture of different probiotic strains.

39. The use or composition for use according to any one of claims 28 to 38, wherein said composition comprises a single probiotic strain.

40. The use or composition for use according to any one of claims 28 to 39, wherein said probiotics comprise Gram-positive bacteria.

41. The use or composition for use according to claim 40, wherein said Gram-positive bacteria are lactic acid bacteria (LAB).

42. The use or composition for use according to claim 41, wherein said LAB is of the Lactococcus genus.

43. The use or composition for use according to claim 42, wherein said LAB of the Lactococcus genus is Lactococcus lactis.

44. The use or composition for use according to claim 43, wherein said Lactococcus lactis is Lactococcus lactis subsp. cremoris.

45. The use or composition for use according to any one of claims 28 to 44, wherein the composition comprising probiotics is for twice-daily administration.

46. The use or composition for use according to any one of claims 28 to 45, wherein the composition comprising probiotics is for administration for a period of at least two weeks.

47. The use or composition for use according to claim 46, wherein the composition comprising probiotics is for administration for a period of about 3 to 5 weeks.

48. The use or composition for use according to any one of claims 28 to 47, wherein the probiotics are present in an amount corresponding to at least about 10⁶ colony-forming units (CFU) in said composition.

49. The use or composition for use according to claim 48, wherein the probiotics are present in an amount corresponding to about 10⁸ to about 10¹² CFU in said composition.

50. The use or composition for use according to any one of claims 28 to 50, wherein the composition comprising probiotics is a liquid composition and comprises a concentration of probiotics of at least about 10⁵ CFU/mL.

51. The use or composition for use according to claim 50, wherein the concentration of probiotics is about 10⁶ to about 10⁸ CFU/mL.

52. The use or composition for use according to claim 50 or 51, wherein the liquid composition comprises a saline buffer.

53. The use or composition for use according to claim 52, wherein said method further comprises resuspending said probiotics in said saline buffer prior to said use.

54. The use or composition for use according to any one of claims 28 to 53, wherein said probiotics and said anti-inflammatory agent are in different compositions.

55. The use or composition for use according to any one of claims 28 to 54, wherein said use improves at least one of the following CRS symptom parameters in said subject: sino-nasal symptom score (SNSS), Sino-nasal Outcome test 22 items (SNOT-22) score and/or peri-operative sinus endoscopy (POSE) score.

56. The use or composition for use according to claim 55, wherein said use improves all of the following CRS symptom parameters in said subject: SNSS, SNOT-22 score, and POSE score.

57. The use or composition for use according to any one of claims 28 to 56, wherein said steroid-resistant CRS is steroid-resistant CRS with nasal polyps.

58. The use or composition for use according to any one of claims 28 to 57, wherein said steroid-resistant CRS is refractory CRS.

59. A kit or package for treating steroid-resistant CRS in a patient, for improving or restoring the response to anti-inflammatory treatment in a steroid-resistant CRS patient, said kit or package comprising a composition comprising an effective amount of probiotics and instructions setting forth the method of any one of claims 1 to 27.

60. The kit or package of claim 59, wherein the composition comprising probiotics is lyophilized, spray-dried or freeze-dried.

61. The kit or package of claim 60, further comprising a solution for reconstituting said composition comprising probiotics.

62. The kit or package of any one of claims 59 to 61, further comprising an anti-inflammatory agent.

63. The kit or package of any one of claims 59 to 62, further comprising a device or system for sinus rinse or sinus irrigation.

64. The kit or package of any one of claims 59 to 62, comprising one of more of the features defined in any one of claims 3 to 27.
